(19) United States
(12) Patent Application Publication
(10) Pub. No.: US 2026/0174321 A1
(43) Pub. Date: Jun. 25, 2026

(54) SPECULUM

(71) Applicant: Gynovent LLC, Cheyenne, WY (US)

(72) Inventors: Jennifer Claman, EL SEGUNDO, CA (US); Michelle Nunez, Dayton, MN (US); Adam Troness, Dellwood, MN (US)

(73) Assignee: Gynovent LLC, Cheyenne, WY (US)

(21) Appl. No.: 19/384,955

(22) Filed: Nov. 10, 2025

Related U.S. Application Data
(60) Provisional application No. 63/737,317, filed on Dec. 20, 2024.

(51) Int. Cl. A61B 1/303; A61B 1/06; A61B 1/32

(52) U.S. Cl. CPC A61B 1/303; A61B 1/0684; A61B 1/32

(57) ABSTRACT

A speculum, including a posterior portion to facilitate gripping thereof, an anterior portion movably connected to the posterior portion, the anterior portion moving in at least one of a first linear direction and a second linear direction, and a light source detachably connected within at least a portion of the anterior portion and at least partially illuminating an area between the posterior portion and the anterior portion in response to being turned on.

### CROSS-REFERENCE TO RELATED APPLICATION

This application incorporates by reference and claims the benefit of priority to U.S. Provisional Application 63/737,317 filed on December 20, 2024.

### BACKGROUND OF THE INVENTION

### 1. Field of the Invention

The present disclosure relates to a speculum, specifically for a vaginal speculum having a light source to provide illumination for improved pelvic examination.

### 2. Description of the Related Art

A vaginal speculum is a medical device used for examination of the vagina and cervix which facilitates access to the uterus via the vagina, and to visualize the cervix and the vagina. When closed, Vaginal Specula can be gently inserted through the introitus (vaginal opening) for the purpose of visualization and examination of the vagina and cervix and to obtain access to the uterus through the cervix.

A vaginal speculum includes a fixed blade (posterior blade) attached to a handle component and a movable blade (anterior blade). There is an “opening mechanism” which allows the back (posterior) and top (anterior) blades to separate. One or both of these blades extends, and advances as needed to provide vaginal and cervical exposure. A locking mechanism such as a ratchet or screw can be used to lock the top and bottom blades in the open position during examination.

FIG. 1 is a perspective view of a prior art vaginal speculum 10. The speculum 10 has a first portion 20 and a second portion 30 movably coupled with each other using at least one hinge 11. The first portion 20 has a handle 21 and a posterior blade 22 extending from the handle 21. The second portion 30 has inspection portion 31 movably coupled with the handle 21 using the hinge 11 and an anterior blade 32 movably coupled with the inspection portion 31. The posterior portion 20 further includes a level 23 and screw 24 for adjusting the distance between the handle 21 and the inspection portion 31.

Some conventional vaginal speculum, such as the vaginal speculum 10, sometimes includes a light source 40 disposed on the first portion 20. The light source 40 is an external light source that often provides inadequate visualization of the vagina or is a trip and fall hazard during use. More specifically, the light source 40 is attached to the inner surface of the first portion 20 and located between the posterior and anterior portions 20, 30. Alternatively, the light source 40 can also be attached or detachably coupled with the posterior blade 22. However, during pelvic examination, fluid such as blood can appear on the inner surface of the first portion 20 to then make contact and damage the light source 40. It is also difficult to remove the light source 40 from the first portion 20 as it is firmly attached to the inner surface of the first portion 20. Further, it is difficult for a physician to insert an examination instrument such as a probe as the light source 40 occupies a significant space between the two blades 22, 32.

Secondly, as illustrated in FIG. 1, a gap 50 exists between the posterior blade 22 and the anterior blade 32. During pelvic examination, vaginal tissue making up the lateral walls of the vagina (vaginal sidewalls) will bulge into the opening preventing adequate examination and/ or sampling. This extra vaginal tissue is not present in all women but has become increasingly more prevalent due to the current obesity epidemic. The redundant tissue is more common in obese women, or those who have had multiple vaginal deliveries. When the patient’s body tissue appears within in the gap 50 the tissue can then be clamped and hurt by the moving blades 22, 32. Also, the patient’s body tissue appearing in the gap 50 may obstruct the physician’s view of the aperture between the blades 22, 32. The health care provider often has to apply additional pressure or expand the device to achieve adequate visualization which can be quite uncomfortable for the patient. Alternately, providers sometimes resort to “home remedies” like placing a glove or other office supply around the available specula. This can be embarrassing for the patient to witness and can lead to increased risk of retained foreign objects if inadvertently left or not completely removed by the provider.

FIG. 2 is a perspective view of another prior art vaginal speculum 10. The posterior portion (posterior blade) 22 further includes a ratchet mechanism 26 for adjusting the distance between the handle 21 and the inspection portion. And a second ratchet mechanism 31 for additional opening or separation of the two blades. However, the vaginal speculum 10 in FIG. 2 illustrates a detachable cord inserted through the handle. This cord is attached to an external power supply and presents a trip and fall hazard for both the patient and the provider. Alternatively, disposable specula use a light coupled to the posterior blade. These lights are very difficult to separate from the speculum and can result in inappropriate disposal into the waste stream. The speculum itself is “biohazard waste” while the battery is “battery waste” if they are not separated, then the device is supposed to be disposed of in “universal” or “black” waste. However, these specula are frequently disposed of in the “biohazard” waste bins posing fine risk and environmental hazards.

However, the use of the above-mentioned the lever 23 with screw and bolt 24 in FIG. 1 and the ratchet mechanism 26 in FIG. 2 create an audible noise which can adversely affect the patient experience. Further, the lever with screw and bolt mechanism in FIG. 1 involves a second separate screw 24 that has the potential to be lost, or become a retained foreign body if not recognized as dislodged, or frequently does not function if the screw mechanism is stripped.

Accordingly, there is a need for a speculum having a light source that will not be contaminated by flowing fluid or blood or tissue during pelvic examination and should be positioned on the speculum to avoid such contamination, and the light source should be easy to remove to facilitate proper disposal.

There is also a need for a speculum that prevents body tissue from appearing between the two blades and obstructing the view of physician and hurting the patient.

There is also a need for a reusable speculum made of plastics that can withstand a high level of disinfection. The present disclosure can be made of a metal strong (such as stainless steel) enough to be capable to assist in patient medical examination, and overlaid by a softer and gentler material to enable a gentler examination. In some embodiments, the speculum system of the present disclosure is made of a strong metal overlaid by a gentler silicone or plastic for a patient medical examination. Here the overlay can adjust to a person’s temperature and allow a gentler examination. In other embodiments, the speculum system is made of hard plastic amenable to high level disinfection or bioplastics with improved disposal profiles.

There is a need for a speculum having a one-handed opening mechanism that does not involve the use of ratchet and detachable screw in order to eliminate the audible sliding sounds and risk of screw malfunction or loss. There is also a need for an opening mechanism that requires less “force” during opening to minimize repetitive motion injury to the provider

There is also the need for a speculum having a wider angle between the posterior (stationary back) blade and the handle. The current 90 degree angle between the posterior (stationary back) blade and the handle causes the speculum to frequently abut against the patient buttocks or encroach upon the anus. The current 90 degree also makes the handle push against any surface that is beneath the patient. This is a frequently encountered problem when the patient is not in a conventional examination table (lithotomy position) as when the patient has need of an examination on such as a gurney or patient emergency room or hospital bed.

There is also the need for a speculum having a curved handle. The current vertical handles encroach upon the patient buttocks and are not ergonomic for the provider.

There is also the need for a speculum having an aerodynamic front entry design that largely avoids the need for the provider to separate the labia during insertion (which is uncomfortable for the patient, especially patients with a history of sexual assault or traumatic pelvic examinations). Current specula the top (anterior) and bottom (posterior) blades leave a gap at the front that can pinch tissue when inserted through the introitus if the labia are not separated by the providers other hand.

### SUMMARY OF THE INVENTION

The present disclosure provides a vaginal speculum.

The foregoing and/or other features and utilities of the present disclosure may be achieved by providing a speculum, including a posterior portion to facilitate gripping thereof, an anterior portion movably connected to the posterior portion, the anterior portion moving in at least one of a first linear direction and a second linear direction, and a light source detachably connected within at least a portion of the anterior portion and at least partially illuminating an area between the posterior portion and the anterior portion in response to being turned on.

The posterior portion includes a curved elongate body, a posterior blade disposed at a first end of the curved elongate body to extend away from the curved elongate body, and a handle disposed at a second end of the curved elongate body to facilitate gripping thereof.

An angle of the curved elongate body between the posterior blade and the handle exceeds ninety degrees.

A rear of the posterior portion and a rear of the anterior portion taper toward a front of the posterior portion and a front of the anterior portion.

The anterior portion includes an anterior blade having an elongate shape, a light attachment portion disposed within at least a portion of the anterior blade to receive the light source therein, and a plurality of side walls, each of the plurality of side walls disposed on at least a portion of a first side of the anterior blade and a second side of the anterior blade, and a first of the plurality of side walls curvedly extending away from the first side of the anterior blade with respect to a first direction and a second of the plurality of side walls curvedly extending away from the second side of the anterior blade with respect to a second direction.

The anterior portion further includes an opened handle disposed on at least a portion of the anterior blade and extending away from the anterior blade with respect to a direction, and a slide lock movably disposed on at least a portion of the opened handle to prevent movement of the anterior portion in a first position and facilitate movement of the anterior portion in at least one of the first linear direction away from the posterior portion and the second linear direction toward the posterior portion.

The speculum further includes a connector connected at a first end to the anterior portion and connected at a second end to the posterior portion.

The anterior portion moves at least partially away from the posterior portion in the first linear direction via the connector, and moves at least partially toward the posterior portion in the second linear direction via the connector.

The connector includes a control wheel movably disposed on at least a portion of the anterior portion.

The anterior portion includes an opened handle disposed on at least a portion of the anterior blade and extending away from the anterior blade with respect to a direction, and a ridged blade disposed on at least a portion of the opened handle and linearly extending away from the opened handle with respect to the direction, the ridged blade to connect to the control wheel to move the anterior portion in at least one of the first linear direction or the second linear direction in response to movement of the control wheel against the ridged blade.

The foregoing and/or other features and utilities of the present disclosure may also be achieved by providing a speculum, including a posterior portion constructed of materials that can withstand a high-level disinfection and facilitates gripping thereof, an anterior portion constructed of materials that can withstand the high-level disinfection and movably connected to the posterior portion, the anterior portion moving in a first rotational direction or a second rotational direction, and a light source detachably connected within at least a portion of the anterior portion and at least partially illuminating an area between the posterior portion and the anterior portion in response to being turned on.

The posterior portion and the anterior portion prevent damage from at least one of bleach, hydrogen peroxide, and a combination of peracetic acid and hydrogen peroxide based on the materials.

The posterior portion and the anterior portion use the materials that include at least one of hard plastic, silicone, polymer, acrylic, ceramics, plastic resins, and metals.

The posterior portion and the anterior portion are constructed of a first material that overlays a second material, the first material is at least one of a plastic, a silicone, a hard plastic, a polymer, an acrylic, a ceramic, and plastic resin, and the second material is a metal.

The foregoing and/or other features and utilities of the present disclosure may also be achieved by providing a speculum, including a posterior portion to facilitate gripping thereof, an anterior portion movably connected to the posterior portion, the anterior portion moving in at least one of a first linear direction and a second linear direction, and a light source detachably connected within at least a portion of the anterior portion and extending from a first end of anterior portion toward a second end of the anterior portion, the light source at least partially illuminating an area between the posterior portion and the anterior portion in response to being turned on.

The light source is at least one of an elongated rod, a square, and a rectangle.

The light source detachably connects within a channel extending from the first end of the anterior portion toward the second end of the anterior portion.

The light source is enclosed within a boundary of the channel after connecting the light source within the channel.

The light source is a micro light-emitting diode (LED).

The light source is detachable from the anterior portion in absence of application of external force from a tool.

### BRIEF DESCRIPTION OF THE DRAWINGS

FIG. 1 is a perspective view of another prior art vaginal speculum.

FIG. 2 is a perspective view of yet another prior art vaginal speculum.

FIG. 3A is a rear perspective view of a speculum in accordance with some embodiments.

FIG. 3B is an elevational top view of the speculum in accordance with some embodiments.

FIG. 3C is an elevational rear view of the speculum in accordance with some embodiments.

FIG. 3D is an elevational side view of the speculum in accordance with some embodiments.

FIG. 4A is a rear perspective view of a posterior portion in accordance with some embodiments.

FIG. 4B is an elevational top view of the posterior portion in accordance with some embodiments.

FIG. 4C is an elevational rear view of the posterior portion in accordance with some embodiments.

FIG. 4D is an elevational side view of the posterior portion in accordance with some embodiments.

FIG. 5A is a rear perspective view of an anterior portion in accordance with some embodiments.

FIG. 5B is an elevational top view of the anterior portion in accordance with some embodiments.

FIG. 5C is an elevational rear view of the anterior portion in accordance with some embodiments.

FIG. 5D is an elevational side view of the anterior portion in accordance with some embodiments.

FIG. 6A is a side perspective view of a speculum in accordance with some embodiments.

FIG. 6B is a side perspective view of a speculum in accordance with some embodiments.

FIG. 6C is a top front perspective view of a speculum in accordance with some embodiments.

FIG. 6D is a bottom front perspective view of a speculum in accordance with some embodiments.

FIG. 6E is a rear perspective view of a speculum in accordance with some embodiments.

FIG. 6F is an elevational top view of a speculum in accordance with some embodiments.

FIG. 7A is an elevational front view of a speculum in accordance with some embodiments.

FIG. 7B is an elevational rear view of a speculum in accordance with some embodiments.

FIG. 8A is side perspective view of a posterior portion of a speculum in accordance with some embodiments.

FIG. 8B is an elevational top view of a posterior portion of a speculum in accordance with some embodiments.

FIG. 8C is a rear perspective view of a posterior portion of a speculum in accordance with some embodiments.

FIG. 9A is side perspective view of an anterior portion of a speculum in accordance with some embodiments.

FIG. 9B is an elevational top view of an anterior portion of a speculum in accordance with some embodiments.

FIG. 9C is a rear perspective view of an anterior portion of a speculum in accordance with some embodiments.

FIG. 10A is a side perspective view of a speculum in accordance with some embodiments.

FIG. 10B is an elevational rear view of a speculum in accordance with some embodiments.

FIG. 11A is side perspective view of a posterior portion of a speculum in accordance with some embodiments.

FIG. 11B is an elevational top view of a posterior portion of a speculum in accordance with some embodiments.

FIG. 11C is a rear perspective view of a posterior portion of a speculum in accordance with some embodiments.

FIG. 12A is side perspective view of an anterior portion of a speculum in accordance with some embodiments.

FIG. 12B is an elevational top view of an anterior portion of a speculum in accordance with some embodiments.

FIG. 12C is a rear perspective view of an anterior portion of a speculum in accordance with some embodiments.

FIG. 13 is a side perspective of a viewing device disposed on a speculum in accordance with some embodiments.

FIG. 14 is a side perspective of a holding device disposed on a speculum in accordance with some embodiments.

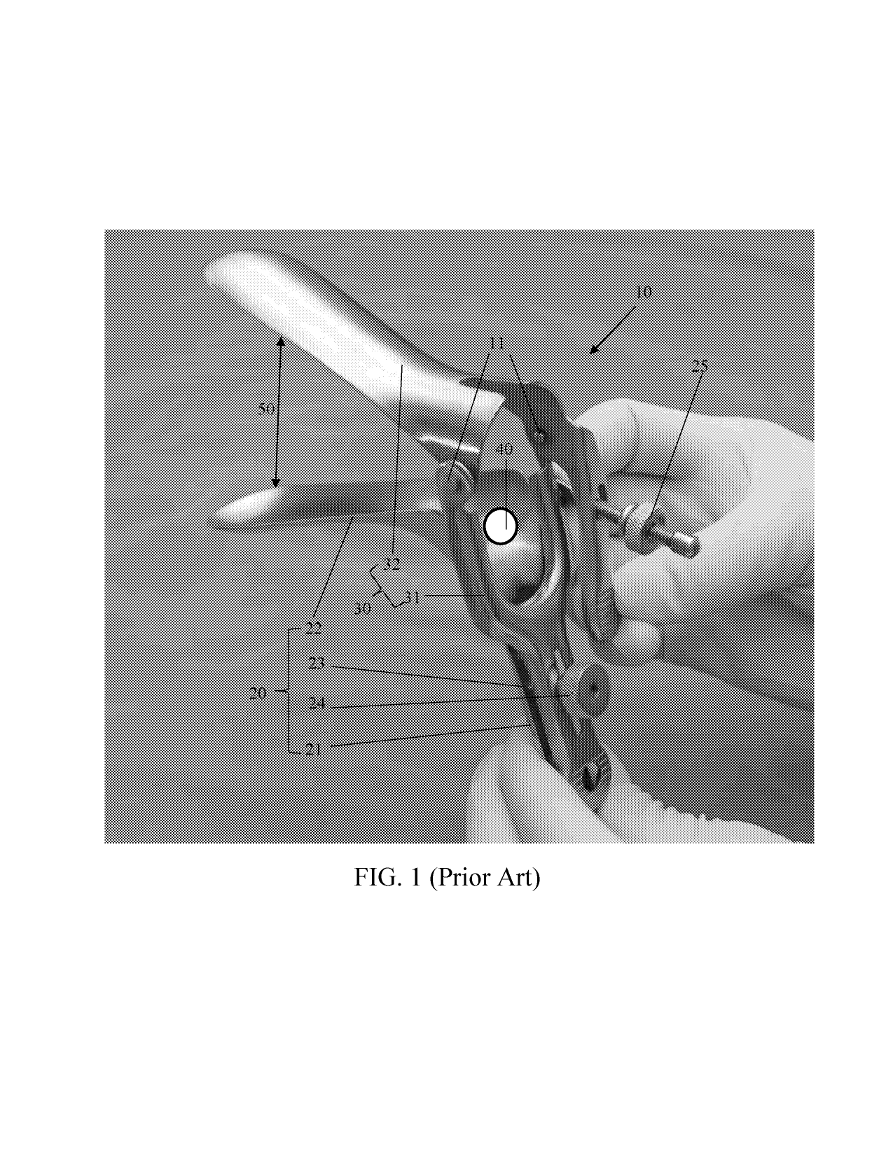

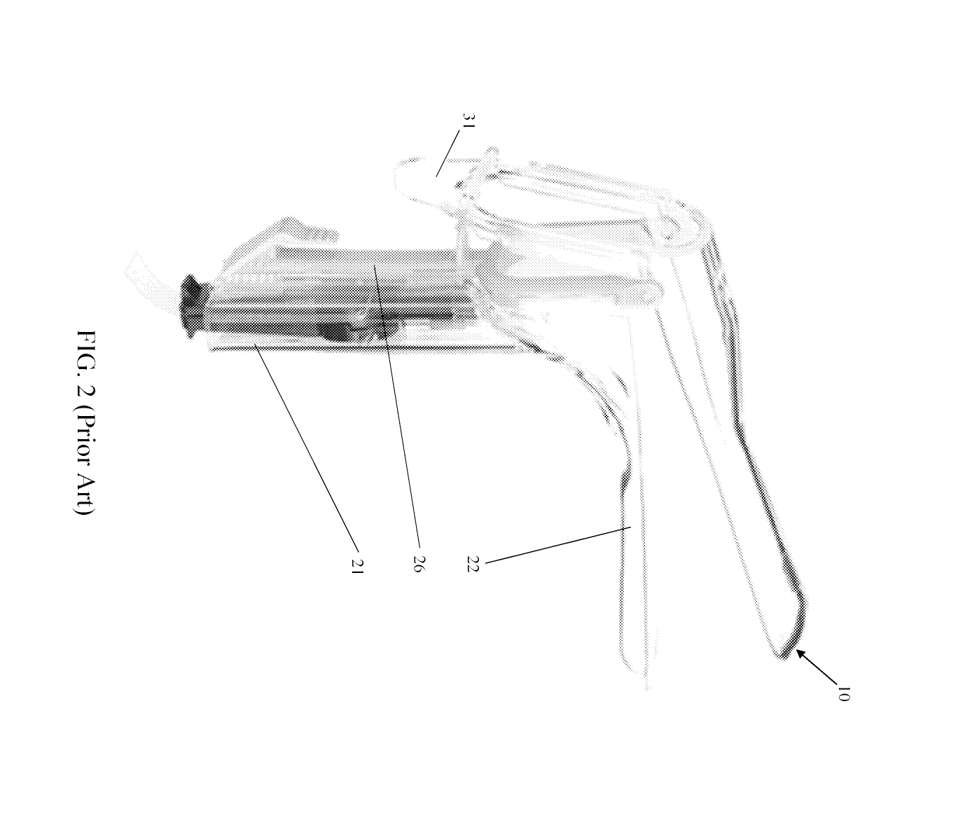

### DETAILED DESCRIPTION OF THE INVENTION

The embodiment and various other embodiments can now be better understood by turning to the following detailed description of the embodiments, which are presented as illustrated examples of the embodiment defined in the claims. It is expressly understood that the embodiment as defined by the claims may be broader than the illustrated embodiments described below. Many alterations and modifications may be made by those having ordinary skill in the art without departing from the spirit and scope of the embodiments.

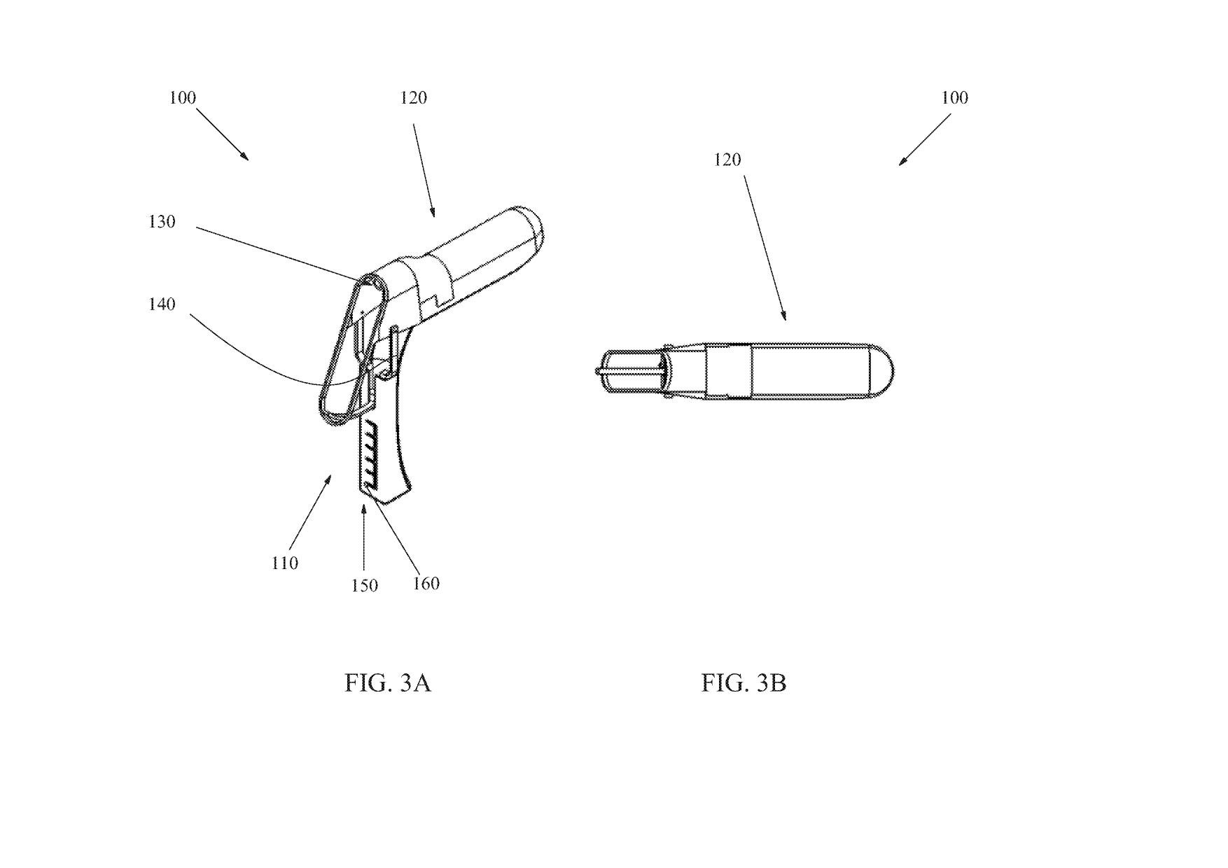

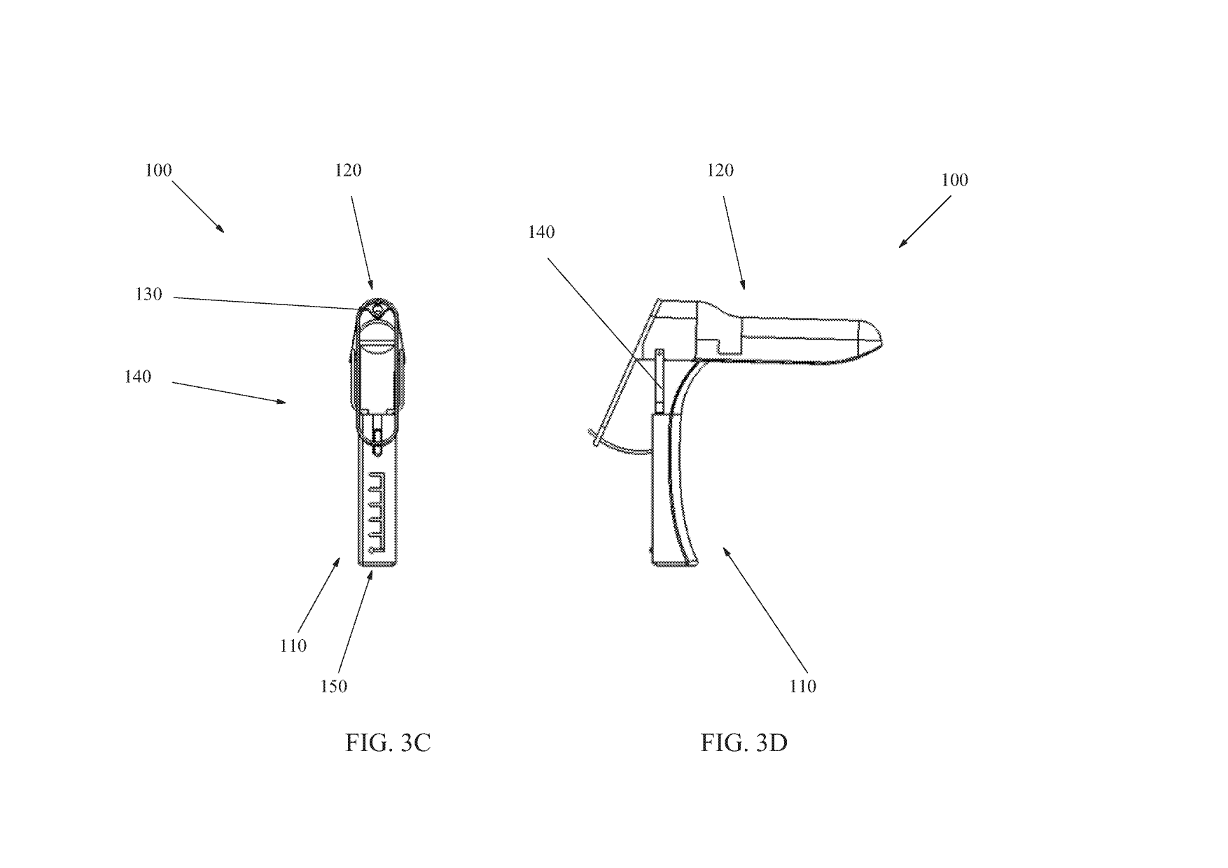

FIG. 3A is a rear perspective view of a speculum 100 in accordance with some embodiments.

FIG. 3B is an elevational top view of the speculum 100 in accordance with some embodiments.

FIG. 3C is an elevational rear view of the speculum 100 in accordance with some embodiments.

FIG. 3D is an elevational side view of the speculum 100 in accordance with some embodiments. The speculum 100 is removably inserted into a vagina of a patient. More specifically, for example, the speculum 100 is inserted in a closed position into the vagina and moved to an opened position to move vaginal walls apart, expanding the vagina, and facilitate viewing of a cervix. In some embodiments, the speculum 100 includes disposable specula made of plastics, reusable specula made of metal, and/or reusable specula made of metal and overlaid with plastics or silicones, or a combination thereof.

In some embodiments, the speculum 100 is similar in size to the speculum commonly referred to as a Graves speculum having two wide blades and suitable for examination of adult patients. In different embodiments, the speculum 100 includes a Bivalve speculum, or a size more typical of a Pediatric speculum, Huffman speculum, Pederson speculum, etc.

Further, the speculum 100 is preferably made of materials that can withstand high-level disinfection that involve the use of chemicals such as a combination of bleach and hydrogen peroxide or a blend of peracetic acid and hydrogen peroxide. Accordingly, the speculum 100 is preferably made of at least one of the following materials: hard plastic (such as methacrylate, polycarbonate, polyvinyl chloride, polyethylene terephthalate glycol, acrylonitrile butadiene styrene) silicone, polymer, acrylic, ceramics, and plastic resins, etc. Furthermore, the speculum 100 includes other materials, such as a “bio-plastic” or “environmentally friendly plastic equivalent” capable of withstanding high level disinfection that is considered more friendly to the environment due to favorable decomposition profile when compared with, for example, polycarbonate or polyvinyl chloride.

Further, for some patients, the speculum 100 made of the above-mentioned materials may not be sturdy and strong enough to open the vaginal canal. Said speculum 100 may require a stronger metal or other material as a base and overlaid by the silicone or softer material. Thus, in different embodiments, the speculum 100 may preferably contain metal, such as stainless steel, and involve over with additional materials other processes including but not exclusive of two-shot molding, co-injection molding, insert molding, hybrid molding, and soft overmolding.

In some embodiments, the speculum 100 includes a posterior portion 110, an anterior portion 120, a light source 130, a connector 140, a sliding track 150, and a lever 160, but is not limited thereto. The posterior portion 110 forms a handle and at least a portion thereof is inserted into the orifice. The anterior portion 120 is removably connected to the posterior portion 110 and at least a portion is inserted into the orifice with a portion of the posterior portion 110. The connector 140 connects the posterior portion 110 (i.e., the handle) to the anterior portion 120. In some embodiments, the connector 140 connects at a first end to the anterior portion 120. Moreover, the anterior portion 120 moves (i.e., shifts, slides) in a first linear direction or a second linear direction in response to an external application of force (e.g., pushing, pulling) that is applied to the connector 140 to separate the anterior portion 120 from the posterior portion 110.

Additionally, in the depicted example of FIGS. 3A through 3D, at least a portion of the connector 140 is at least partially disposed within the posterior portion 110. The connector 140 is movably disposed within the posterior portion 110. More specifically, a second end of the connector 140 moves (i.e., slides) in a first linear direction or a second linear direction from disposed within the posterior portion 110 (i.e., stored within the posterior portion 110) to at least partially extracted from the posterior portion 110. Conversely, the second end of the connector 140 moves in the second linear direction or the first linear direction from at least partially extracted from the posterior portion 110 to disposed within the posterior portion. In other words, the connector 140 moves at least partially in and out of the posterior portion 110. Therefore, the anterior portion 120 moves in the first linear direction or the second linear direction in response to movement of the connector 140.

Referring to FIGS. 3A and 3C, the sliding track 150 is disposed on at least a portion of the posterior portion 110. In particular, the sliding track 150 forms a plurality of grooves on the posterior portion 150. Additionally, the lever 160 is movably disposed (i.e., slidably) on at least a portion of the sliding track 150. Furthermore, the lever 160 is connected to at least a portion of the connector 140. Accordingly, the connector 140 moves in response to movement of the lever 160, such as an external application of force (e.g., pushing, pulling) on the lever 160. For example, the connector 140 moves in the first linear direction in response to moving (i.e., sliding) the lever 160 along a portion of the sliding track 150 in the first linear direction. Conversely, the connector 140 moves in the second linear direction opposite to the first linear direction in response to moving the lever 160 along a portion of the sliding track 150 in the second linear direction. Stated differently, sliding the lever 160 along the sliding track 150 separates or opens the anterior portion 120 away from the posterior portion 110. Thus, sliding the lever 160 creates a gap between the posterior portion 110 and the anterior portion 120 to facilitate visualization. It is important to note that moving the connector 140 also slightly moves the anterior portion 120 by providing additional opening with a rotational component that is sometimes necessary for optimal visualization of the cervix.

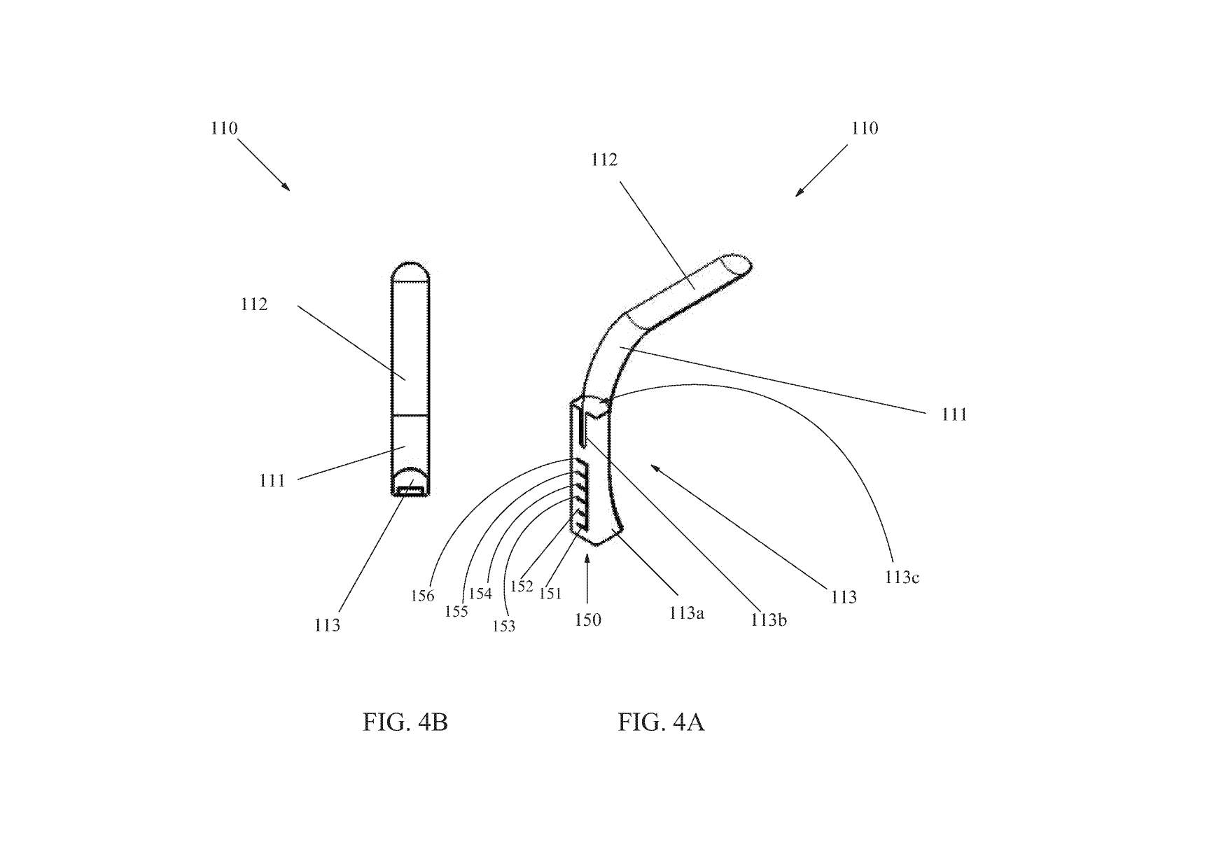

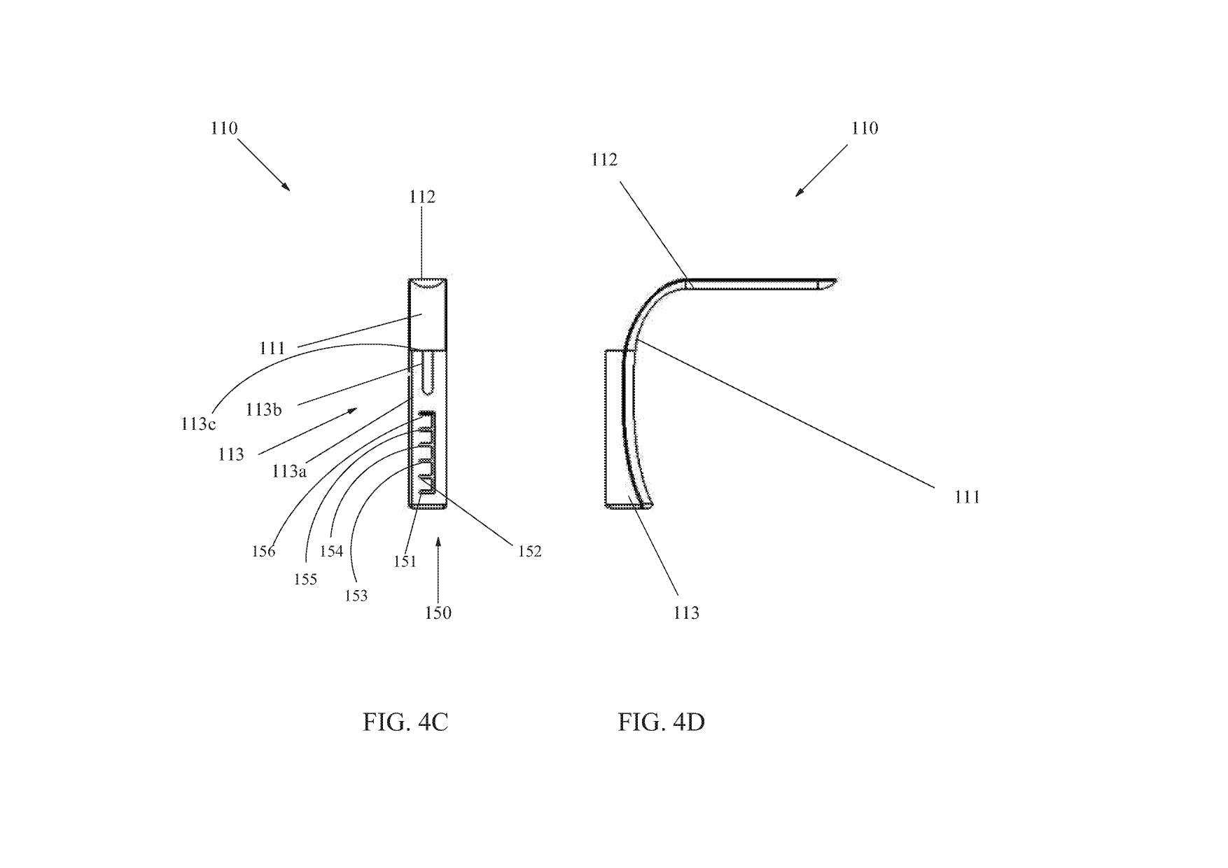

FIG. 4A is a rear perspective view of a posterior portion 110 in accordance with some embodiments.

FIG. 4B is an elevational top view of the posterior portion 110 in accordance with some embodiments.

FIG. 4C is an elevational rear view of the posterior portion 110 in accordance with some embodiments.

FIG. 4D is an elevational side view of the posterior portion 110 in accordance with some embodiments. Referring to FIGS. 4A through 4D, in some embodiments, the posterior portion 110 includes a curved elongate body 111, a posterior blade 112, and a handle 113, but is not limited thereto. The curved elongate body 111 is constructed in an arcuate shape.

The posterior blade 112 is disposed at a first end of the curved elongate body 111. The posterior blade 112 extends away from the first end of the curved elongate body 111 with respect to a linear direction. In application, the posterior blade 112 facilitates insertion thereof into the orifice (e.g., vagina) of the patient. The handle 113 is disposed at a second end of the curved elongate body 111. In the depicted example of FIGS. 4A and 4D, the handle 113 is larger (i.e., greater size in length, width, depth) than the curved elongate body 111. However, in different embodiments, the handle 113 is similar in size to the curved elongate body 111, depending on construction. The posterior blade 112 connects to the handle 113 with a greater than ninety degree (90°) angle. For example, in some embodiments, the posterior blade 112 connects to the handle 113 between 110 to 130 degrees. The angle facilitates visualization during operation by preventing tissue impediment, such as from buttocks of the patient, or from hitting a bed or gurney in those patients not being examined in standard lithotomy position. In other words, the greater than 90 degree angle of the posterior blade 112 and/or the handle 113 prevent the buttocks from obstructing view of the vagina and the cervix. It also helps when the patient is on a gurney. The handle 113 is curved to prevent abutting against the buttocks and a rectum of the patient.

In some embodiments, the handle 113 includes a handle body 113a, a slit 113b, and a connector receiving channel 113c, but is not limited thereto. The handle body 113a is elongate and facilitates gripping thereof. In other words, the handle body 113a is used to grip the posterior portion 110. The slit 113b is disposed on at least a portion of the handle body 113a, such as a first end of the handle body 113a and extends toward a center of the handle body 113a. Referring to FIGS. 3A, 3C, and 3D, the slit 113b receives at least a portion of the connector 140 therein. Stated differently, at least a portion of the connector 140 protrudes through the slit 113b. The connector receiving channel 113c is disposed within an interior of the handle body 113a. That is, the connector receiving channel 113c forms a hollow interior of the handle body 113a. In the depicted example of FIGS. 4A and 4D, the connector receiving channel 113c receives at least a portion of the handle body 113a therein. In particular, a second end of the handle body 113a is disposed within the connector receiving channel 113c and contacts an end of the connector receiving channel 113c. Accordingly, at least a portion of the handle body 113a is disposed within the connector receiving channel 113c. Furthermore, the connector receiving channel 113c receives the connector 140 therein and facilitates movement of the connector 140 in the first linear direction or the second linear direction in response to movement of the lever 160.

Referring to FIGS. 4A and 4C, the sliding track 150 is disposed on at least a portion of the handle body 113a. The sliding track 150 includes a first groove 151, a second groove 152, a third groove 153, a fourth groove 154, a fifth grove 155, and a sixth groove 156, but is not limited thereto. The first groove 151 is disposed proximal to the second end of the handle body 113a at a first distance less than each distance of the second groove, 152, the third groove 153, the fourth groove 154, the fifth groove 155, and the sixth groove 156 to the second end of the handle body 113a. On the other hand, the sixth groove 156 is disposed distal from the second end of the handle body 113a at a distance greater than each distance of the first groove 151, the second groove, 152, the third groove 153, the fourth groove 154, and the fifth groove 155 to the second end of the handle body 113a. Each succeeding groove of the sliding track 150 after the first groove 151 increases in distance from the second end of the handle body 113a. The first groove 151, the second groove, 152, the third groove 153, the fourth groove 154, the fifth groove 155, and/or the sixth groove 156 (collectively, the grooves) receive the lever 160 therein in response to moving the lever 160 along the sliding track 160 and moving the lever 160 into the first groove 151, the second groove, 152, the third groove 153, the fourth groove 154, the fifth groove 155, and/or the sixth groove 156. In this manner, the first groove 151, the second groove, 152, the third groove 153, the fourth groove 154, the fifth groove 155, and/or the sixth groove 156 prevent further movement along the sliding track 150 into a different groove, while the lever 160 remains inserted within at least one of the grooves.

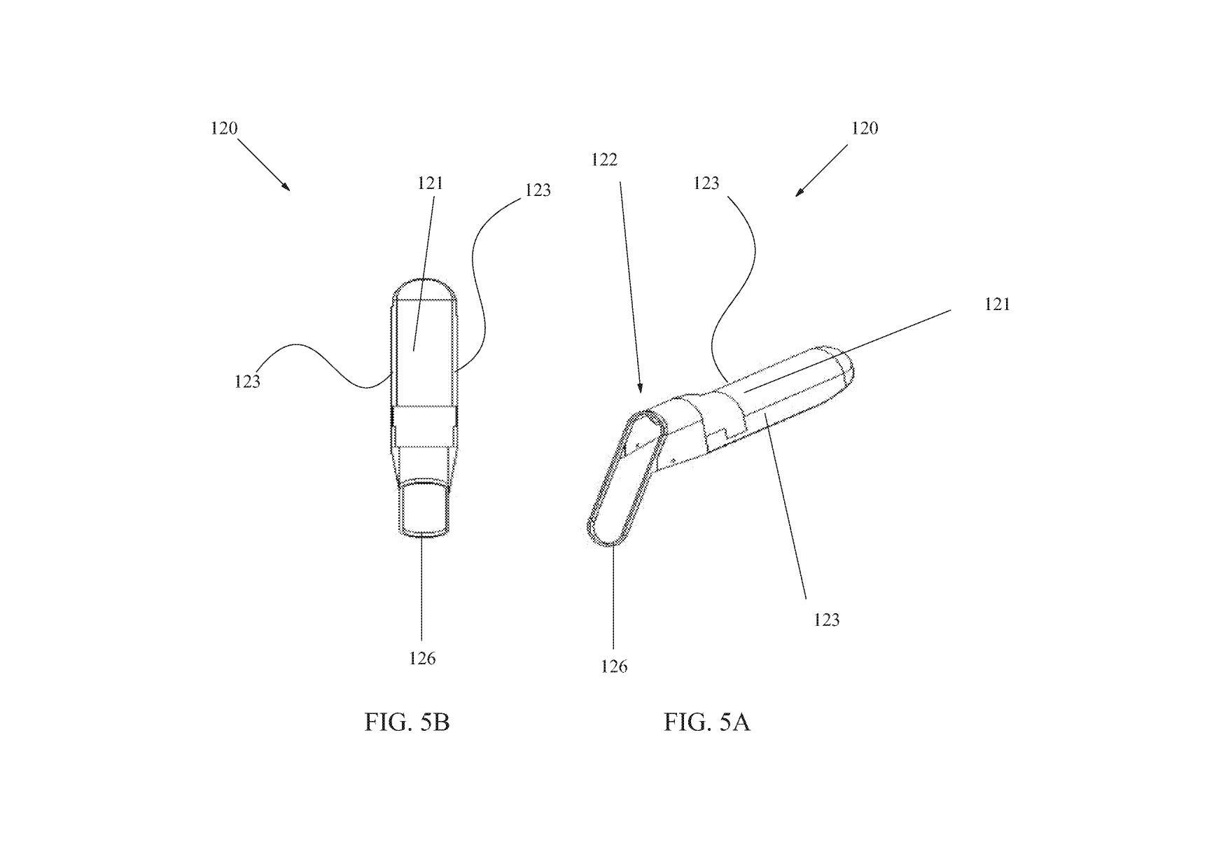

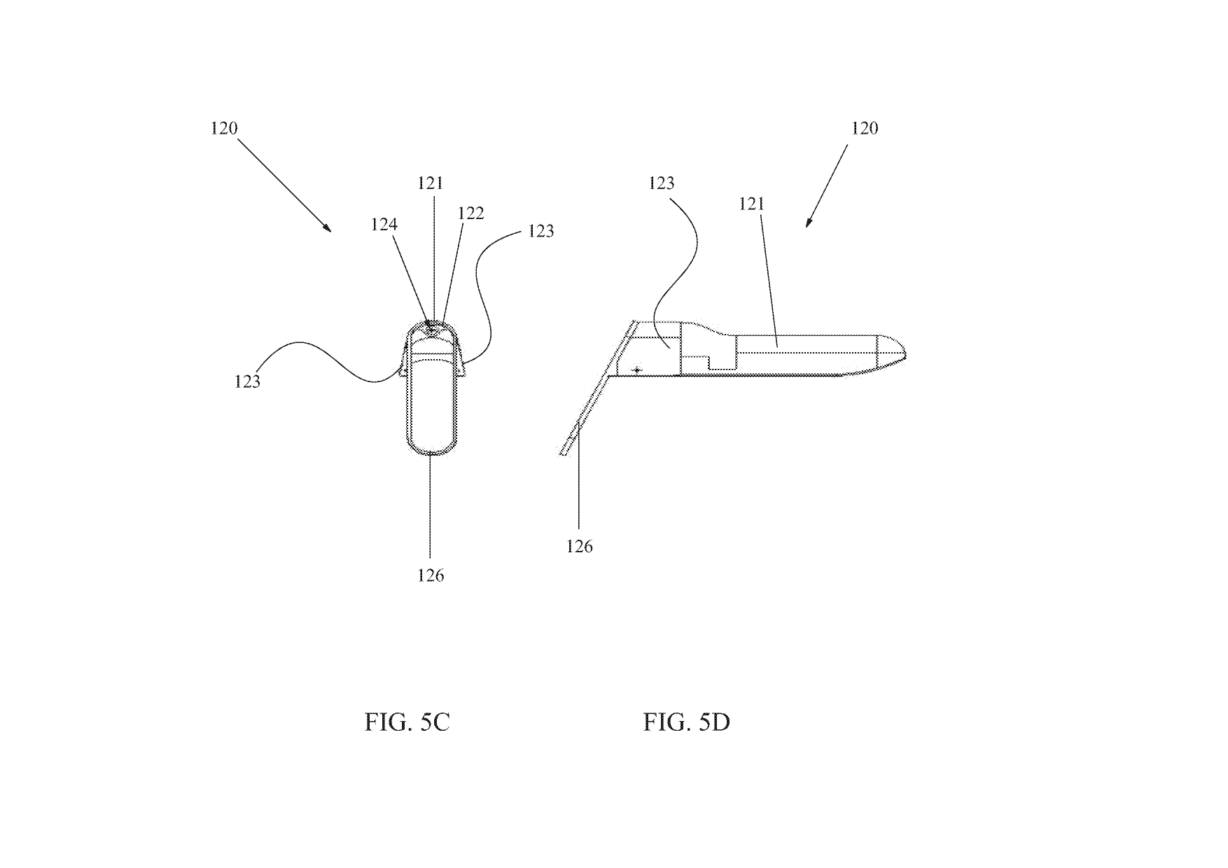

FIG. 5A is a rear perspective view of a anterior portion 120 in accordance with some embodiments.

FIG. 5B is an elevational top view of the anterior portion 120 in accordance with some embodiments.

FIG. 5C is an elevational rear view of the anterior portion 120 in accordance with some embodiments.

FIG. 5D is an elevational side view of the anterior portion 120 in accordance with some embodiments. Referring to FIGS. 5A through 5D, in some embodiments, the anterior portion 120 includes an anterior blade 121, a light attachment portion 122, a plurality of side walls 123, a channel 124, and a connector gap 126, but is not limited thereto. The anterior blade 121 is constructed with an elongate shape. In some embodiments, the anterior blade 121 has a similar size (e.g., length, width, depth) as a size (e.g., length, width, depth) of the posterior blade 112. However, in different embodiments, the size of the anterior blade 121 is larger or smaller than the size of the posterior blade 112 based on different construction. In application, the anterior blade 121 facilitates insertion thereof into the orifice (e.g., vagina) of the patient. Collectively, the posterior blade 112 and the anterior blade 121 facilitate insertion into the vagina of the patient. Furthermore, the anterior blade 121 moves at least a portion of the vagina in response to moving in the first rotational direction or the second rotational direction, and/or the first linear direction or the second linear direction, as described above. In particular, the anterior blade 121 expands the vagina in response to moving in the first rotational direction or the second rotational direction, and/or the first linear direction or the second linear direction, such that movement of the anterior blade 121 within the vagina facilitates access therein and access to the cervix of the patient, such as during medical operations (e.g., surgery). The posterior blade 112 and/or the anterior blade 121 are preferably two smooth and curved metal or plastic blades. The user clinician inserts the speculum 100 into the vagina in the closed position with the anterior blade 121 and the posterior blade 112 on top of each other, not separated, and are then gently opened to hold the vaginal walls apart, providing a clear view of the cervix. The posterior blade 112 and/or the anterior blade 121 preferably have rounded and polished edges to minimize discomfort and risk of tissue injury during insertion and removal of the speculum 100.

In the depicted example of FIGS. 5A and 5C, the light attachment portion 122 is disposed on at least a portion of the anterior blade 121 and/or within a portion of the anterior blade 121. The plurality of side walls 123 are disposed on at least a portion of the anterior blade 121. More specifically, a first side wall of the plurality of side walls 123 is disposed on at least a portion of a first side of the anterior blade 121 and extends away from the first side of the anterior blade 121 with respect to a first direction (i.e., a first angle from a vertex of the anterior blade 121), and a second side wall of the plurality of side walls 123 is disposed on at least a portion of a second side of the anterior blade 121 and extends away from the second side of the anterior blade 121 with respect to a second direction (i.e., a second angle from a vertex of the anterior blade 121) different from the first direction. However, in some embodiments, the first side wall of the plurality of side walls 123 and the second side wall of the plurality of side walls 123 extend away from the first side of the anterior blade 121 and the second side of the anterior blade 121 in a same direction.

Referring to FIGS. 3A, 3C, 3D, 5A, 5C, and FIG. 5D, each of the plurality of side walls 123 extend away from the anterior blade 121 toward the posterior blade 112. As such, the plurality of side walls 123 at least partially obstruct an area between the plurality of side walls 123 and the posterior blade 112. Accordingly, the plurality of side walls 123 prevent an external object, such as body tissue within the vagina of the patient from entering the area between the plurality of side walls 123 and/or the posterior blade 112. In conventional specula, the anterior blade and/or posterior blade are flat or have slight curvature similar to a spoon. In contrast, the anterior blade 121 is curved like “C” shape, such that the plurality of side walls 123 are sloped to facilitate retraction during insertion into the vagina. During medical use, the plurality of side walls 123 are sloped to facilitate retraction of the vagina without moving the posterior blade 112 and/or the anterior blade 121. In other words, the plurality of side walls 123 separate tissue within the vagina during insertion of the speculum 100 without moving (i.e., separating) the anterior blade 121 away from the posterior blade 112.

As described above, the anterior portion 120 includes the plurality of side walls 123 and extend away from the anterior blade 121. Alternatively, and/or in addition thereto, in different embodiments, the posterior portion 110 includes the plurality of side walls 123. For example, the plurality of side walls 123 are disposed on a first side of the posterior blade 112 and a second side of the posterior blade, and each of the plurality of side walls 123 on the posterior blade 112 extend away from the posterior blade 112 toward the anterior blade 121. Similarly, the plurality of side walls 123 at least partially obstruct the area between the plurality of side walls 123 and the anterior blade 121. Accordingly, the plurality of side walls 123 prevent the external object, such as body tissue within the vagina of the patient from entering the area between the plurality of side walls 123 and/or the anterior blade 121.

The channel 124 is disposed within at least a portion of the light attachment portion 122. The channel 124 extends from a first end of the light attachment portion 122 toward a second end of the light attachment portion 122. Also, the channel 124 extends from a first end of the anterior blade 121 toward a second end of the anterior blade 121. In other words, the channel 124 is elongate and, in some embodiments, is cylindrical in shape. However, in different embodiments, the shape of the channel 124 is rectangular, triangular, pentagonal, hexagonal, heptagonal, or octagonal.

In some embodiments, the connector gap 126 is disposed at a first end of the anterior blade 121 and extends angularly away from the first end of the anterior blade 121. Referring to FIGS. 3A through 3D and 5A through 5D, the connector gap 126 receives at least a portion of the connector 140 therein. Moreover, the connector gap 126 facilitates movement of the anterior portion 120 in the first rotational direction or the second rotational direction, as will be discussed further below.

In some embodiments, the light source 130 is a micro light-emitting diode (LED). However, in different embodiments, the light source 130 is an incandescent light, a fluorescent bulb, a xenon blub, and the like. In some embodiments, the light source 130 is an elongated rod. In different embodiments, the light source 130 includes mini LED, organic LED, and other light sources that are small, energy-efficient, and can be manually switched on and off. Further, in different embodiments, the light source 130 can be configured to be removably attached to the anterior blade 121. In other words, the posterior blade 112 and/or the anterior blade 121 in different embodiments may be free of the light attachment portion 122 and can be detachably coupled with the light source 130. In different embodiments, the anterior blade 121 is separate, independent, detachably coupled light source 130 that is inserted.

The light source 130 detachably connects (i.e., inserts) within the channel 124 of the light attachment portion 122 and extends from the first end of the channel 124 toward the second end of the channel 124. It is important to note that the light source 130 is detachable from the channel 124 in absence of an application of external force from a tool (e.g., screwdriver, wrench, hammer, etc.). In other words, the light source 130 is detachable using only hands or other limb of a user. The light source 130 is enclosed within a boundary of the channel 124 after connecting the light source 130 within the channel 124. The light source 130 at least partially illuminates an area between the posterior blade 112 (i.e., posterior portion 110) and the anterior blade 121 (i.e., anterior portion 120) in response to being turned on. The light source 130 provides illumination for example, the vaginal cavity and specifically illuminates the vaginal walls and cervix for better visualization during examination. Conversely, the light source 130 is prevented from illuminating in response to being turned off.

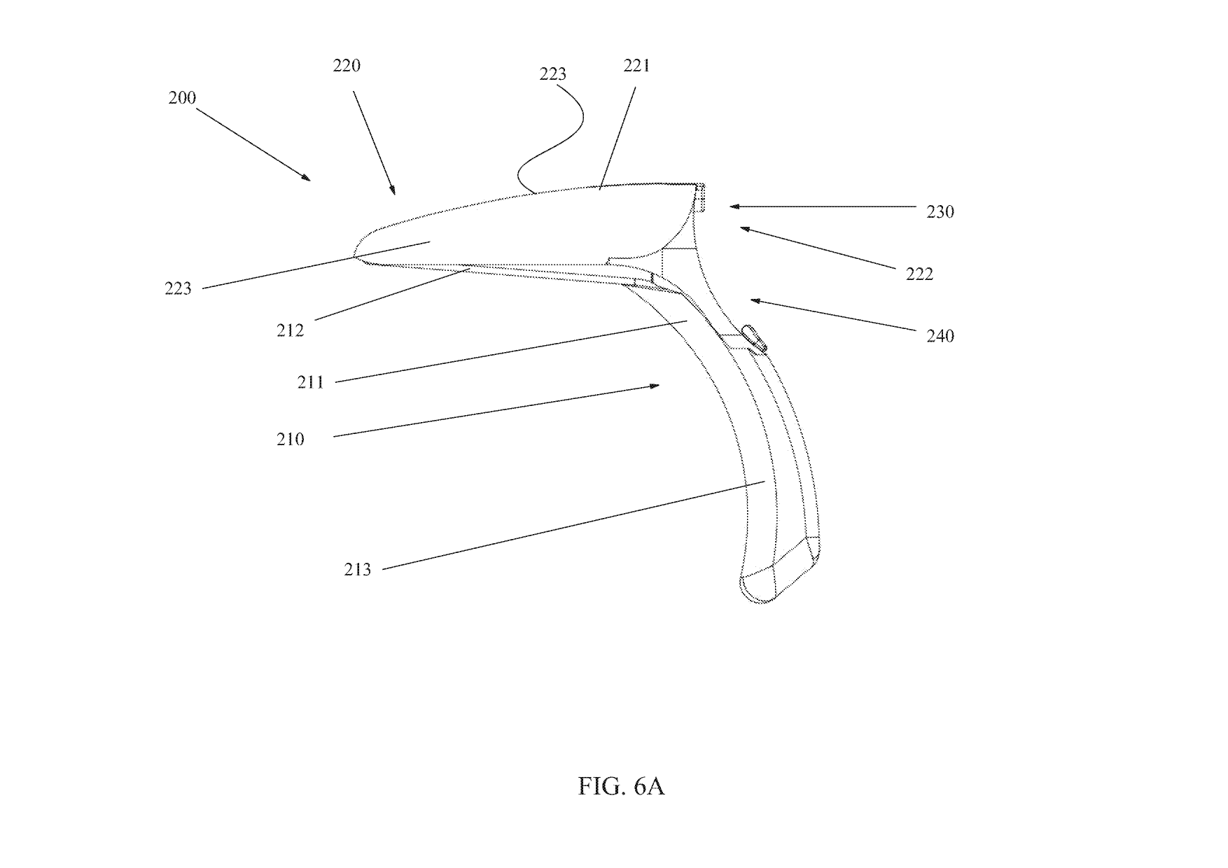

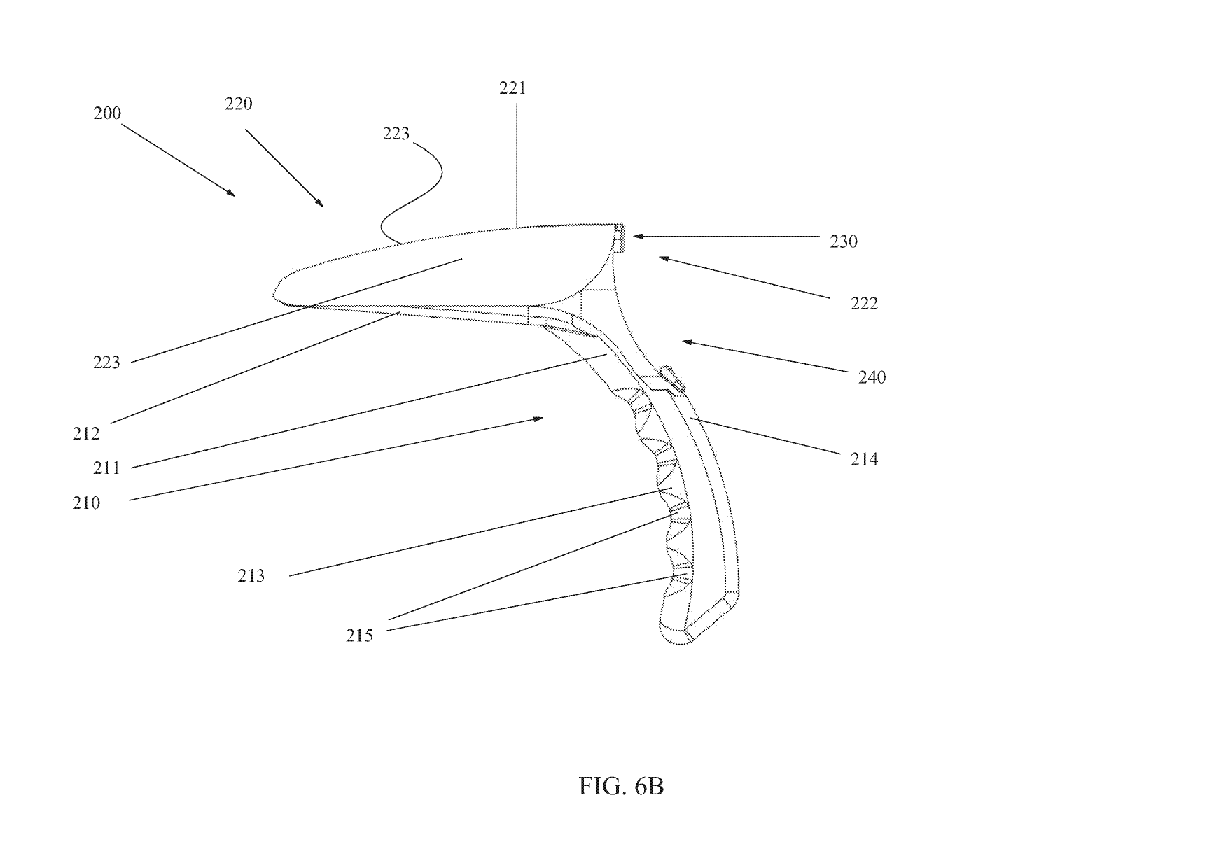

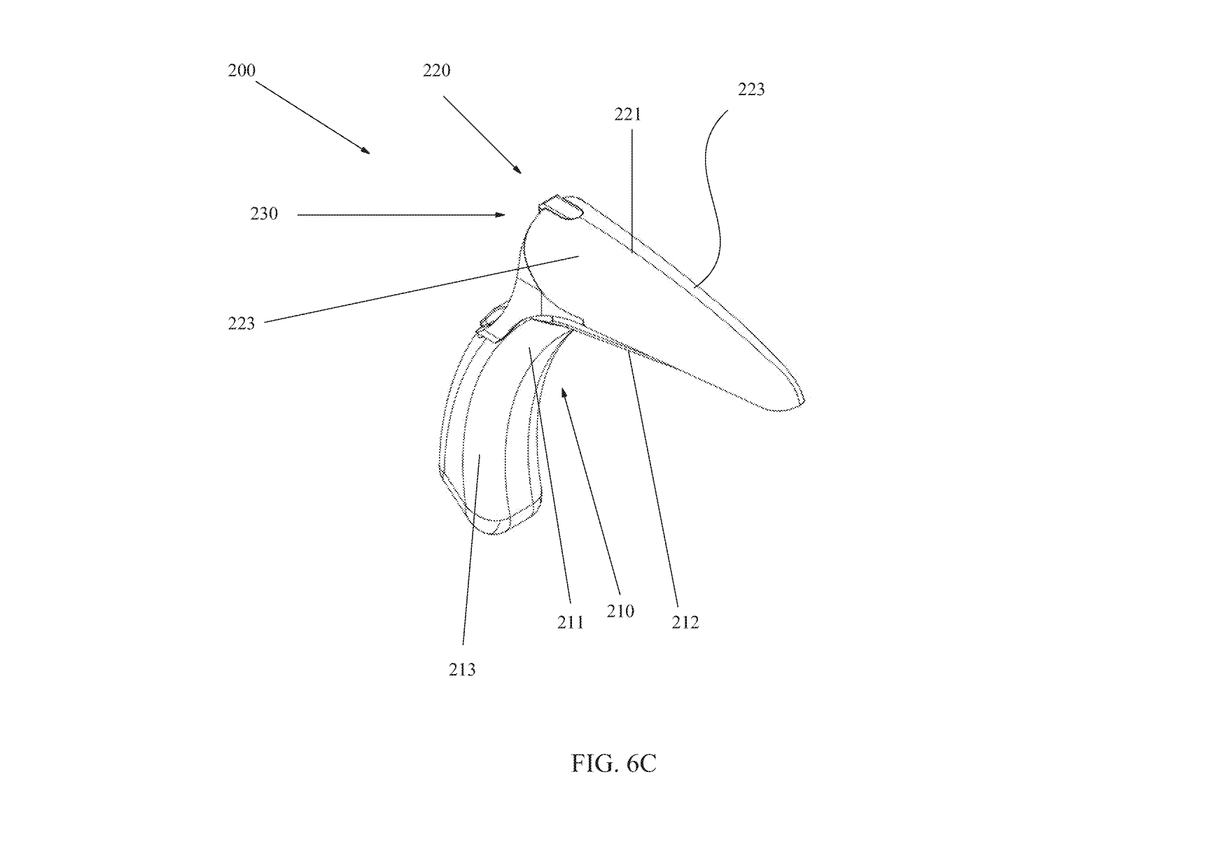

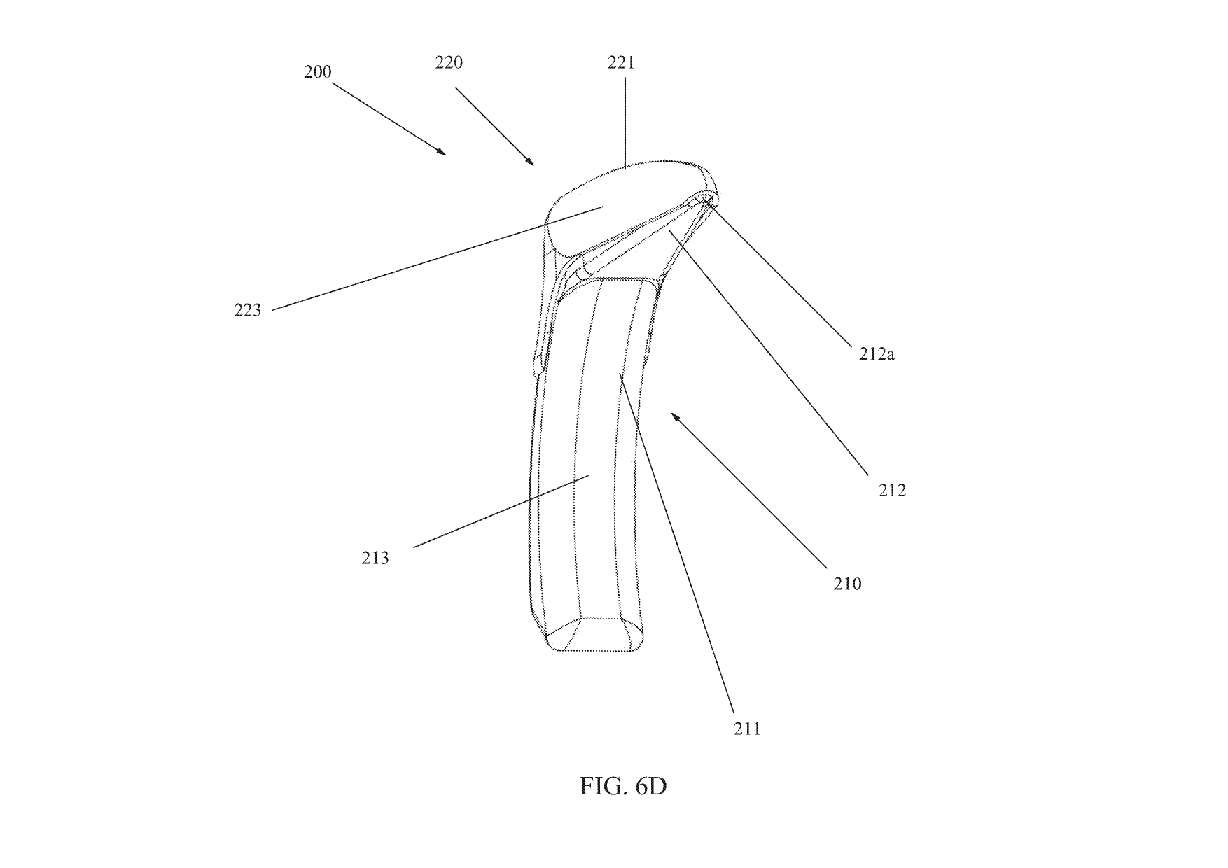

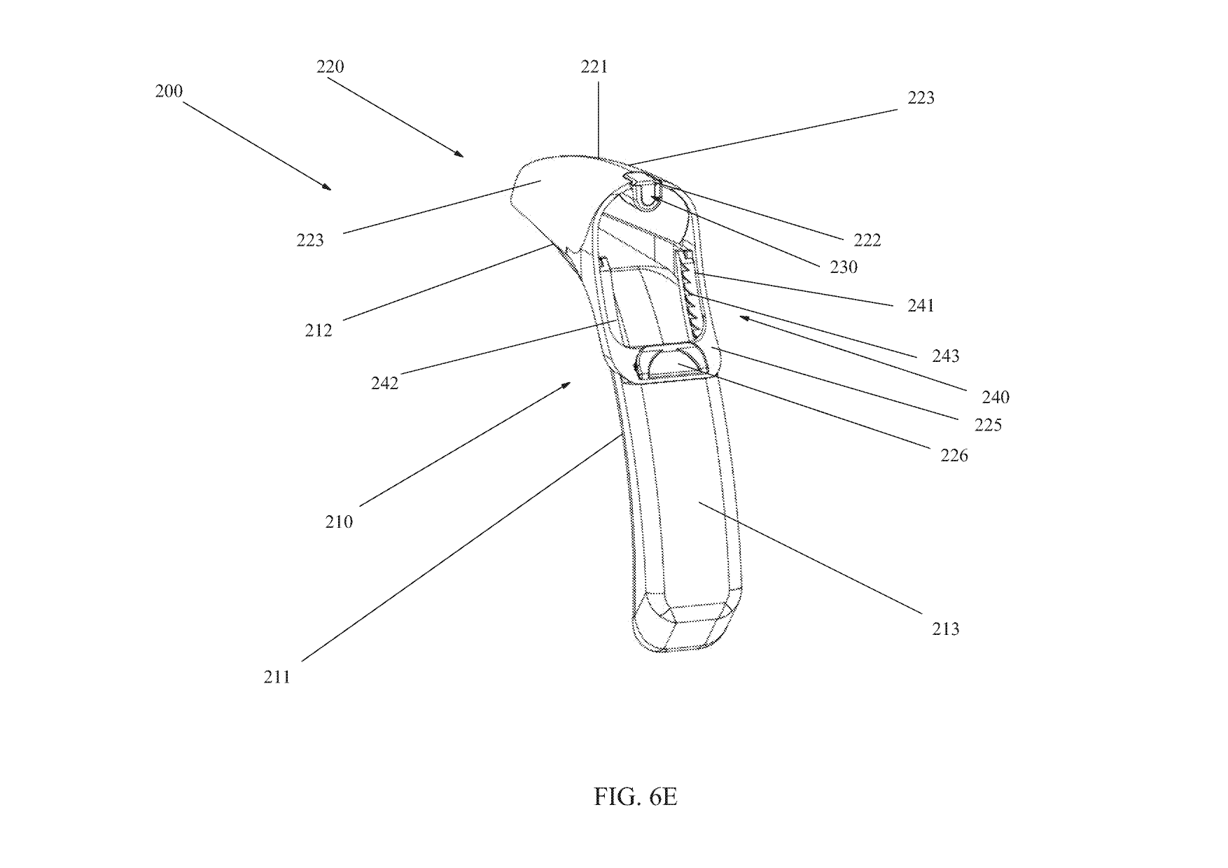

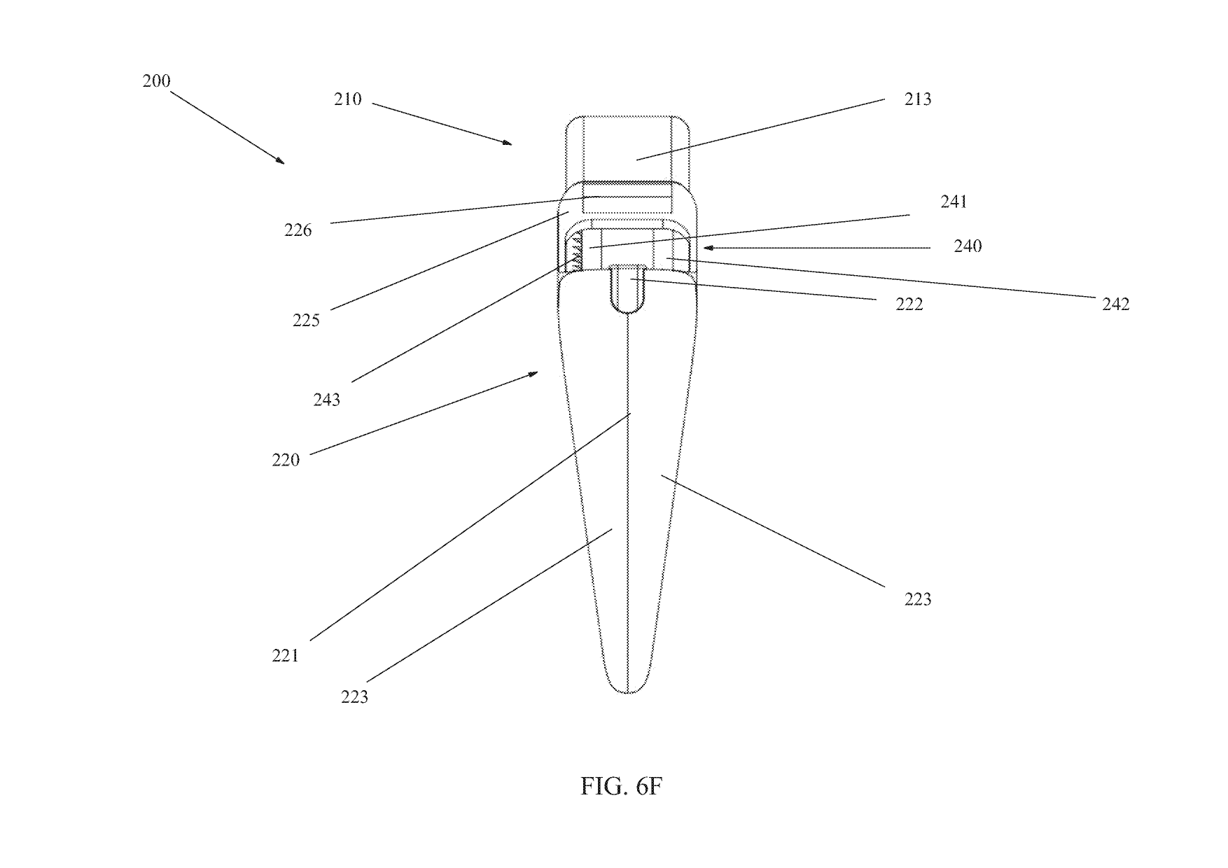

FIG. 6A is a side perspective view of a speculum 200 in accordance with some embodiments.

FIG. 6B is a side perspective view of a speculum 200 in accordance with some embodiments.

FIG. 6C is a top front perspective view of a speculum 200 in accordance with some embodiments.

FIG. 6D is a bottom front perspective view of a speculum 200 in accordance with some embodiments.

FIG. 6E is a rear perspective view of a speculum 200 in accordance with some embodiments.

FIG. 6F is an elevational top view of a speculum 200 in accordance with some embodiments. The speculum 200 may implement or be implemented by aspects of the speculum 100 as described with reference to FIGS. 3A through 5D. Similar to the speculum 100, the speculum 200 is a device that generally is used for medical examination of a patient. The speculum 200 is removably inserted into a vagina of a patient. More specifically, for example, the speculum 200 is inserted in a closed position into the vagina and moved to an opened position to move vaginal walls apart, expanding the vagina, and facilitate viewing of a cervix. In some embodiments, the speculum 200 includes disposable specula made of plastics, reusable specula made of metal, and/or reusable specula made of metal and overlaid with plastics or silicones, or a combination thereof.

Further, the speculum 200 is preferably made of materials that can withstand high-level disinfection that involve the use of chemicals such as a combination of bleach and hydrogen peroxide or a blend of peracetic acid and hydrogen peroxide. Accordingly, the speculum 200 is preferably made of at least one of the following materials: hard plastic (such as methacrylate, polycarbonate, polyvinyl chloride, polyethylene terephthalate glycol, acrylonitrile butadiene styrene) silicone, polymer, acrylic, ceramics, and plastic resins, etc.

Further, for some patients, the speculum 200 made of the above-mentioned materials may not be sturdy and strong enough to open the vaginal canal. Said speculum 200 may require a stronger metal or other material as a base and overlaid by the silicone or softer material. Thus, in different embodiments, the speculum 200 may preferably contain metal, such as stainless steel, and involve over with additional materials other processes including but not exclusive of two-shot molding, co-injection molding, insert molding, hybrid molding, and soft overmolding.

Referring to FIGS. 6A through 6F, in some embodiments, the speculum 200 includes a posterior portion 210, an anterior portion 220, a light source 230, and a connector 240, but is not limited thereto. The posterior portion 210 forms a handle and at least a portion thereof is inserted into the orifice. The anterior portion 220 is removably connected to the posterior portion 210 and at least a portion is inserted into the orifice with a portion of the posterior portion 210. The connector 240 connects the posterior portion 210 to the anterior portion 220. Moreover, the anterior portion 220 moves (i.e., slides, shifts) in a first linear direction away from the posterior portion 210 or a second linear direction toward the posterior portion 210 in response to an external application of force (e.g., pushing, pulling).

In some embodiments, the posterior portion 210 includes a curved elongate body 211, a posterior blade 212, a handle 213, a thumb recess 214, and a plurality of finger recesses 215, but is not limited thereto. The curved elongate body 211 is constructed in an arcuate shape (e.g., a C-shape). In contrast, prior speculum handles do not have any curved shape, let alone, a C-shape.

The posterior blade 212 is disposed at a first end of the curved elongate body 211. The posterior blade 212 extends away from the first end of the curved elongate body 211 with respect to a linear direction. In application, the posterior blade 212 facilitates insertion thereof into the orifice (e.g., vagina) of the patient. The handle 213 is disposed at a second end of the curved elongate body 211. The handle 213 is elongate and facilitates gripping thereof. In other words, the handle 213 is used to grip the posterior portion 210. In the depicted example of FIG. 6A, the handle 213 is larger (i.e., greater size in length, width, depth) than the curved elongate body 211. However, in different embodiments, the handle 213 is similar in size to the curved elongate body 211, depending on construction. The posterior blade 212 connects to the handle 213 with a greater than ninety degree (90°) angle. For example, in some embodiments, the posterior blade 212 connects to the handle 213 between 110 to 130 degrees. The angle facilitates visualization during operation by preventing tissue impediment, such as from buttocks of the patient. In other words, the increased angle between the posterior blade 212 and/or the handle 213 prevent the buttocks from obstructing view of the vagina and the cervix. It also helps when the patient is on a gurney. The handle 213 is curved to prevent abutting against the buttocks and a rectum of the patient.

In the depicted example of FIG. 6B, the thumb recess 214 is disposed on a first side of the handle 213 and the plurality of finger recesses 215 are disposed on a second side of the handle 213 opposite with respect to the first side. In particular, the thumb recess 214 receives a thumb therein and each of the plurality of finger recesses 215 receives at least one finger therein. That is, the thumb recess 214 receives a thumb therein and each of the plurality of finger recesses 215 improve a friction level during gripping. In some embodiments, the plurality of finger recesses 215 include five recesses. However, in different embodiments, a number of the plurality of finger recesses 215 is less than five or more than five.

Referring to FIGS. 6A through 6F, in some embodiments, the anterior portion 220 includes an anterior blade 221, a light attachment portion 222, a plurality of side walls 223, and a channel 224, but is not limited thereto. The anterior blade 221 is constructed with an elongate shape. Referring to FIGS. 6C through 6F, it is important to note that the anterior blade 221 tapers from a first end (i.e., rear) toward a second end (i.e., front) opposite to the first end. Similarly, the posterior blade 212 tapers from a first end (e.g., rear) toward a second end (i.e., front) opposite to the first end. As such, the second end of the posterior blade 212 and the second end of the anterior blade 221 are narrower with respect to the first end of the posterior blade 212 and the first end of the anterior blade 221, respectively. In this manner, the anterior blade 221 improves comfort for the patient during insertion into the vagina, being narrower at the initial insertion point, while the larger (i.e., wider) first end, back of the speculum 200 improves visualization of the vagina and the cervix during operation by a medical professional by providing a wide viewing area and for passage of instruments as for a biopsy while at the same time, minimizing patient discomfort by narrowing the majority of the speculum 200 inside the vagina.

Referring to FIG. 6D, in some embodiments, the posterior blade 212 includes a rounded tip 212a, but is not limited thereto. The rounded tip 212a curves in a direction. In particular, and preferably, the rounded tip 212a is disposed to curve toward the anterior blade 221. In some embodiments, the anterior blade 221 has a similar size (e.g., length, width, depth) as a size (e.g., length, width, depth) of the posterior blade 212. However, in different embodiments, the size of the anterior blade 221 is larger or smaller than the size of the posterior blade 212 based on different construction. In application, the anterior blade 121 facilitates insertion thereof into the orifice (e.g., vagina) of the patient. Collectively, the posterior blade 212 and the anterior blade 221 facilitate insertion into the vagina of the patient. Furthermore, the anterior blade 221 moves at least a portion of the vagina in response to moving in the first linear direction or the second linear direction, as described above. In particular, the anterior blade 221 expands the vagina in response to moving in the first linear direction or the second linear direction, such that movement of the anterior blade 221 within the vagina facilitates access therein and access to the cervix of the patient, such as during medical operations (e.g., surgery). The posterior blade 212 and/or the anterior blade 221 are preferably two smooth and curved metal or plastic blades. The user clinician inserts the posterior blade 212 and/or the anterior blade 221 into the vagina while closed and then gently moves the anterior blade 221 away from (i.e., separates) the posterior blade 212 to hold the vaginal walls apart, providing a clear view of the cervix. The posterior blade 212 and/or the anterior blade 221 preferably have rounded and polished edges to minimize discomfort and risk of tissue injury during insertion and removal of the speculum 200. Therefore, the posterior blade 212 and/or the anterior blade 221 come together at the front, tapered portion with no exposed edges to provide aerodynamic entry into the vagina and minimize the need to spread the labia during insertion and minimize the risk of pinching tissue.

In the depicted example of FIGS. 6A and 6B, the light attachment portion 222 is disposed on at least a portion of the anterior blade 221 and/or within a portion of the anterior blade 221. The plurality of side walls 223 are disposed on at least a portion of the anterior blade 221. More specifically, a first side wall of the plurality of side walls 223 is disposed on at least a portion of a first side of the anterior blade 221 and extends away from the first side of the anterior blade 221 with respect to a first direction (i.e., a first angle from a vertex of the anterior blade 221), and a second side wall of the plurality of side walls 223 is disposed on at least a portion of a second side of the anterior blade 221 and extends away from the second side of the anterior blade 221 with respect to a second direction (i.e., a second angle from a vertex of the anterior blade 221) different from the first direction. However, in some embodiments, the first side wall of the plurality of side walls 223 and the second side wall of the plurality of side walls 223 extend away from the first side of the anterior blade 221 and the second side of the anterior blade 221 in a same direction. Referring again to FIGS. 6A and 6B, each of the plurality of side walls 223 extend away from the anterior blade 221 toward the posterior blade 212. As such, the plurality of side walls 223 at least partially obstruct an area between the plurality of side walls 223 and the posterior blade 212. Accordingly, the plurality of side walls 223 prevent an external object, such as body tissue within the vagina of the patient from entering the area between the plurality of side walls 223 and/or the posterior blade 212. In conventional specula, the anterior blade 221 and/or the posterior blade 212 are flat or have slight curvature similar to a spoon. In contrast, the anterior blade 221 is curved like “C” shape, such that the plurality of side walls 223 are sloped to facilitate retraction during insertion into the vagina. During medical use, the plurality of side walls 223 are sloped to facilitate retraction of the vagina without moving the posterior blade 212 and/or the anterior blade 221. In other words, the plurality of side walls 223 separate tissue within the vagina during insertion of the speculum 200 without moving (i.e., separating) the anterior blade 221 away from the posterior blade 212.

As described above, the anterior portion 220 includes the plurality of side walls 223 and extend away from the anterior blade 221. Alternatively, and/or in addition thereto, in different embodiments, the posterior portion 210 includes the plurality of side walls 223. For example, the plurality of side walls 223 are disposed on a first side of the posterior blade 212 and a second side of the posterior blade, and each of the plurality of side walls 223 on the posterior blade 212 extend away from the posterior blade 212 toward the anterior blade 221. Similarly, the plurality of side walls 223 at least partially obstruct the area between the plurality of side walls 223 and the anterior blade 221. Accordingly, the plurality of side walls 223 prevent the external object, such as body tissue within the vagina of the patient from entering the area between the plurality of side walls 223 and/or the anterior blade 221.

The channel 224 is disposed within at least a portion of the light attachment portion 222. The channel 224 extends from a first end of the light attachment portion 222 toward a second end of the light attachment portion 222. Also, the channel 224 extends from a first end of the anterior blade 221 toward a second end of the anterior blade 221. In other words, the channel 224 is elongate and, in some embodiments, is cylindrical in shape. However, in different embodiments, the shape of the channel 224 is rectangular, triangular, pentagonal, hexagonal, heptagonal, or octagonal.

In some embodiments, the light source 230 is a micro light-emitting diode (LED). However, in different embodiments, the light source 230 is an incandescent light, a fluorescent bulb, a xenon blub, and the like. In some embodiments, the light source 230 includes an elongated rod, a square, and a rectangle, but is not limited thereto. In different embodiments, the light source 230 includes mini LED, organic LED, and other light sources that are small, energy-efficient, and can be manually switched on and off. Further, in different embodiments, the light source 230 can be configured to be removably attached to the anterior blade 221. In other words, the posterior blade 212 and/or the anterior blade 221 in different embodiments may be free of the light attachment portion 222 and can be detachably coupled with the light source 230. In different embodiments, the anterior blade 221 is separate, independent, detachably coupled light source 230 that is inserted.

The light source 230 detachably connects (i.e., inserts) within the channel 224 of the light attachment portion 222 and extends from the first end of the channel 224 toward the second end of the channel 224. It is important to note that the light source 230 is detachable from the channel 224 in absence of an application of external force from a tool (e.g., screwdriver, wrench, hammer, etc.). In other words, the light source 230 is detachable using only hands or other limb of a user. The light source 230 is enclosed within a boundary of the channel 224 after connecting the light source 230 within the channel 224. The light source 230 at least partially illuminates an area between the posterior blade 212 (i.e., posterior portion 210) and the anterior blade 221 (i.e., anterior portion 220) in response to being turned on. The light source 230 provides illumination for example, the vaginal cavity and specifically illuminates the vaginal walls and cervix for better visualization during examination. Because the light source is on the anterior blade, it is not obscured by blood, tissue or other body fluid running out of the vagina along the posterior blade. Additionally, because the light is inserted along the anterior blade, it provides more direct illumination of the cervix and distal vagina. Conversely, the light source 230 is prevented from illuminating in response to being turned off.

Referring to FIGS. 6E and 6F, the connector 240 includes a first track 241, a second track 242, and a plurality of locking indents 243, but is not limited thereto. The first track 241 is disposed on at least a portion of a first side of the curved elongate body 211. The second track 242 is disposed on at least a portion of a second side of the curved elongate body 211 opposite to the first side of the curved elongate body 211. Moreover, the plurality of locking indents 243 are disposed on at least a portion of the first track 241. The first track 241 and the second track 242 receive a corresponding connecting rail therein. The connecting rails are disposed on at least a portion of the anterior portion 220.

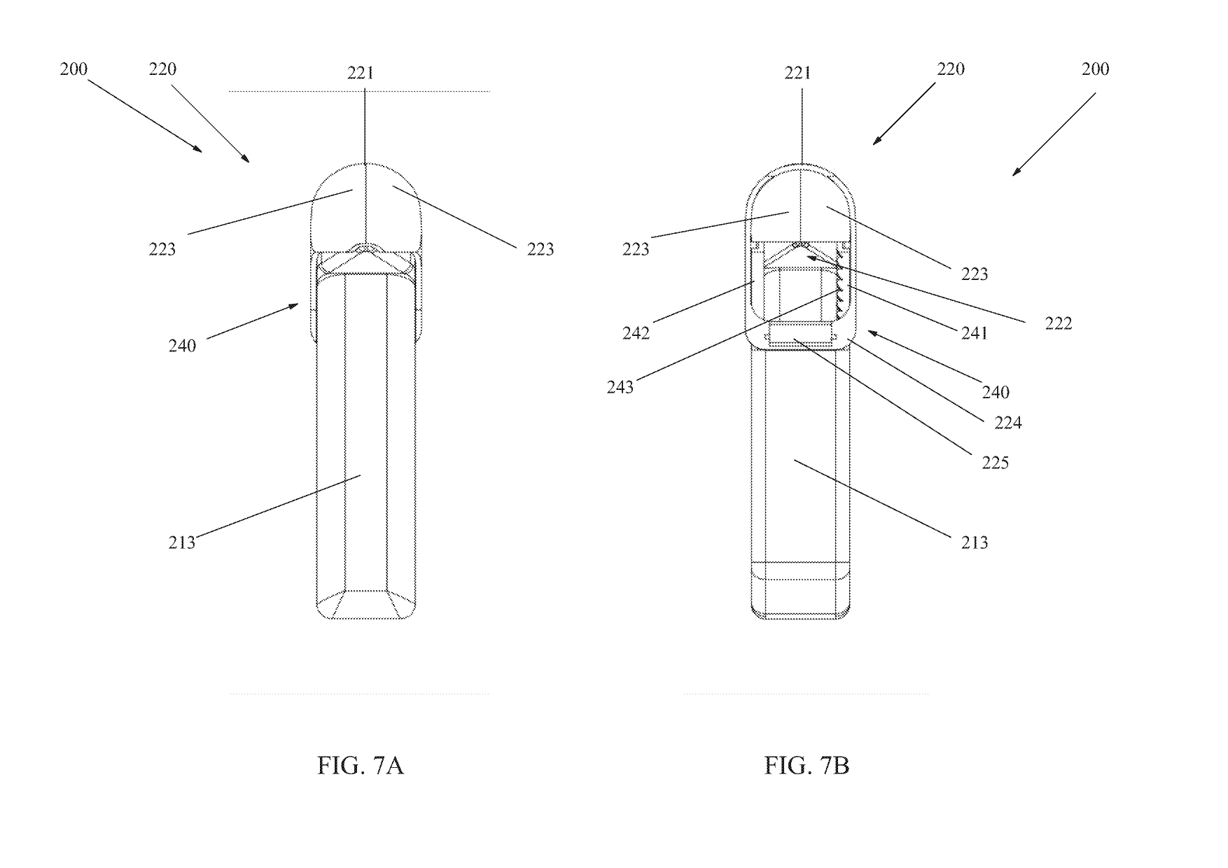

FIG. 7A is an elevational front view of a speculum 200 in accordance with some embodiments.

FIG. 7B is an elevational rear view of a speculum 200 in accordance with some embodiments. In the depicted example of FIGS. 6E through 7B, the anterior portion 220 further includes an opened handle 225 and a slide lock 226, but is not limited thereto. The opened handle 225 is angularly disposed at a rear end of the anterior blade 221. The opened handle 225 extends away from the rear end of the anterior blade 221 with respect to a direction. The opened handle 225 includes an opening to facilitate viewing an area between the posterior blade 212 and/or the anterior blade 211. As such, the opened handle 225 facilitates viewing of an interior of the vagina and/or the cervix while inserted into the vagina. The slide lock 226 is movably (i.e., slidably) disposed on at least a portion of the opened handle 225. The slide lock 226 prevents movement of the anterior portion 220 in response to moving (e.g., locking) within at least one of the plurality of locking indents 243. To illustrate via an example, the anterior portion 220 moves (i.e., slides) in the first linear direction away from the posterior portion 210 in response to an application of force (e.g., pushing) against the opened handle 225. Thereafter, the slide lock 226 is moved (i.e., slid) in a third linear direction (i.e., to the right with respect to FIGS. 6E and 7B), such that the slide lock 226 moves between at least two of the plurality of locking indents 243. The plurality of locking indents 243 prevent the slide lock 226 and the anterior portion 220 from further movement in the first linear direction or the second linear direction. Subsequently, moving the slide lock 226 in a fourth linear direction (i.e., to the left with respect to FIGS. 6E and 7B), such that the slide lock 226 moves away from, and disengages from two of the plurality of locking indents 243. As such, the anterior portion 220 is movable again based on an application of force in the first linear direction or the second linear direction toward the posterior portion 210.

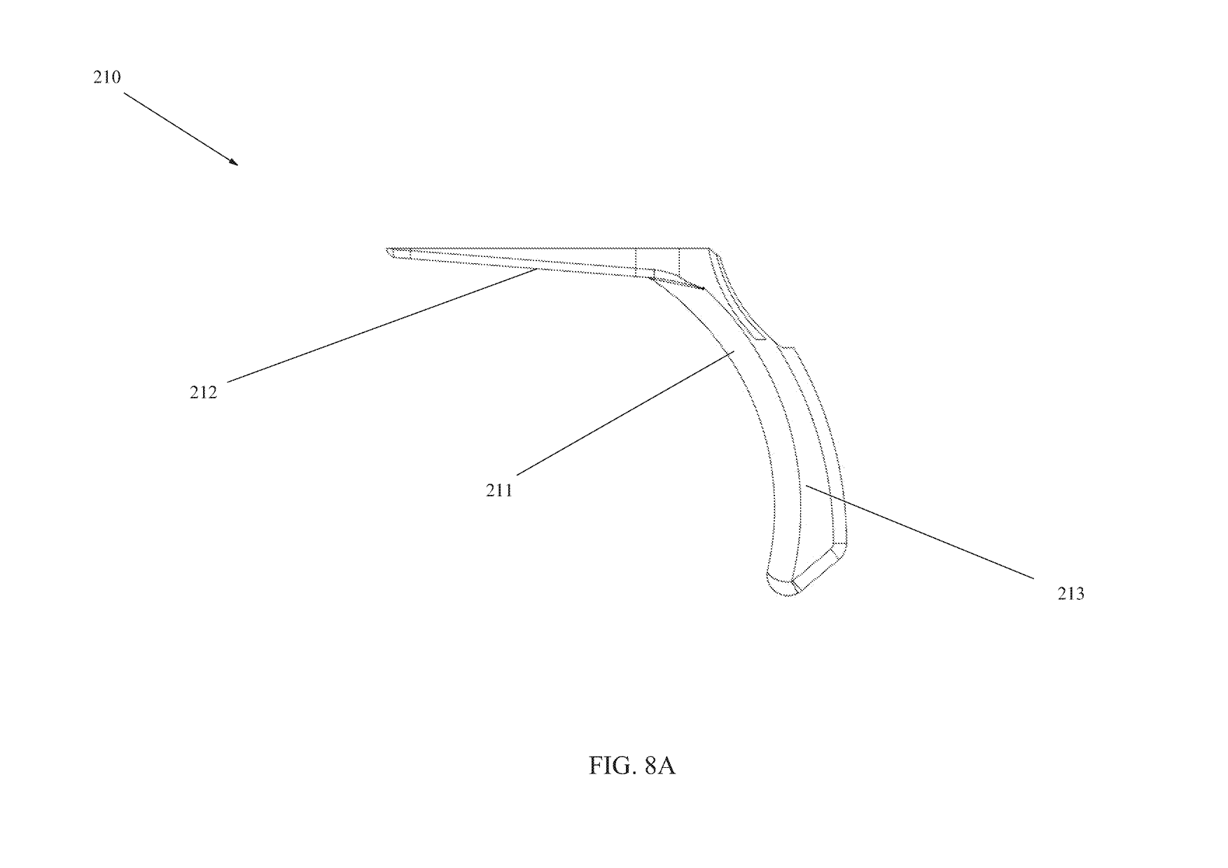

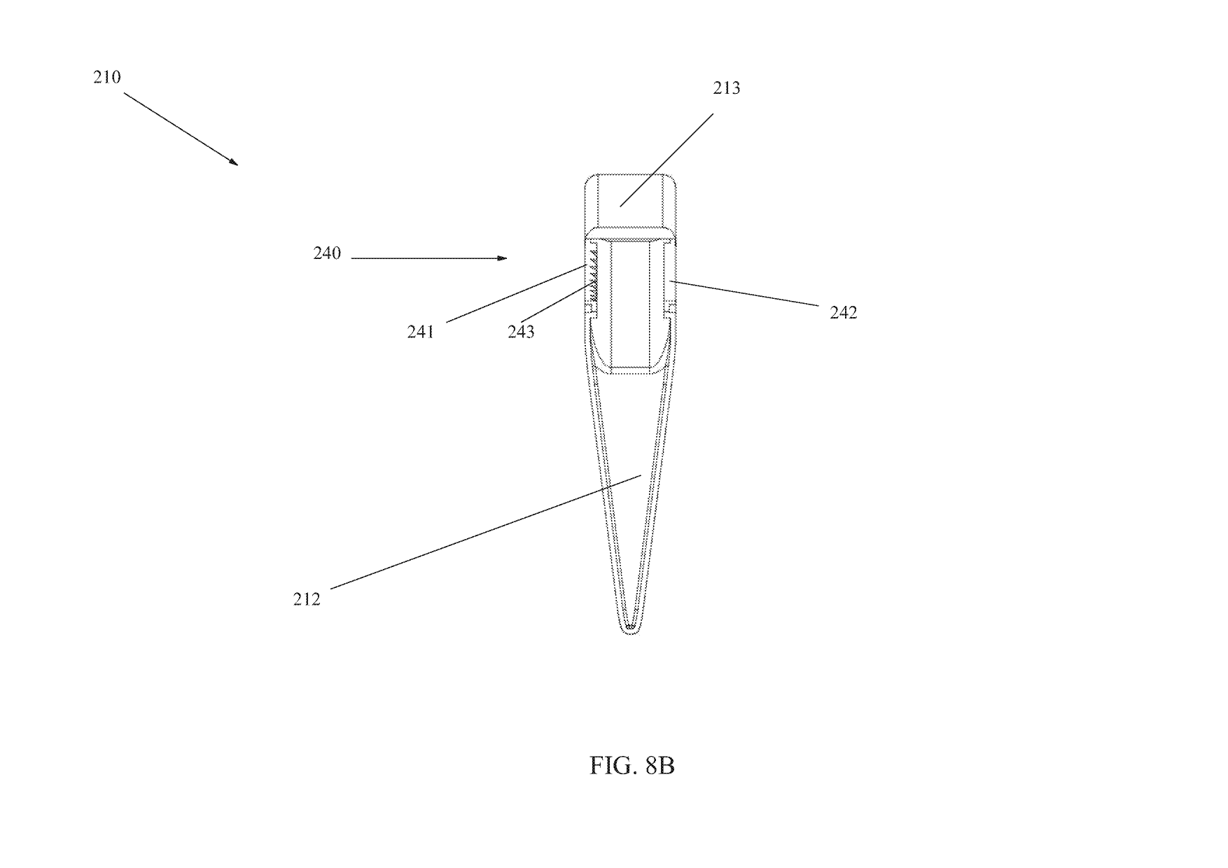

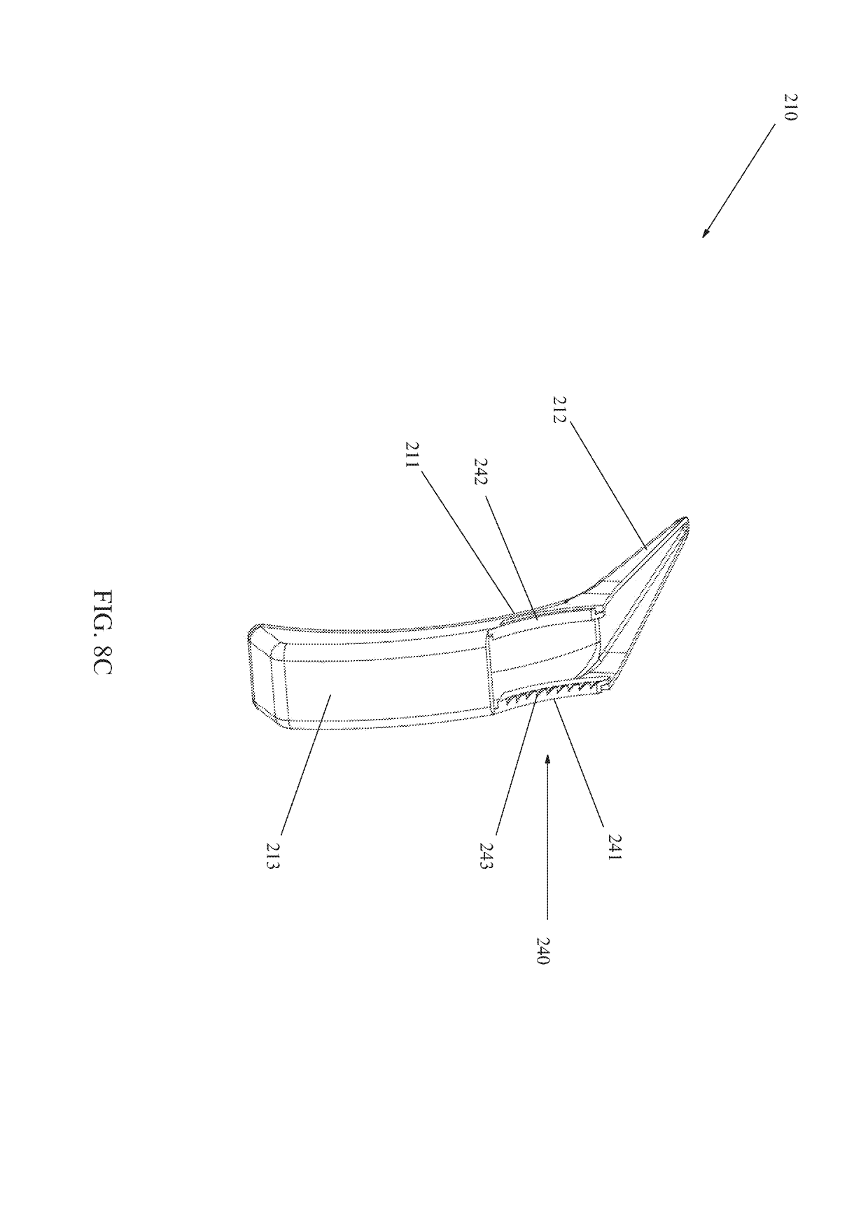

FIG. 8A is side perspective view of a posterior portion 210 of a speculum 200 in accordance with some embodiments.

FIG. 8B is an elevational top view of a posterior portion 210 of a speculum 200 in accordance with some embodiments.

FIG. 8C is a rear perspective view of a posterior portion 210 of a speculum 200 in accordance with some embodiments.

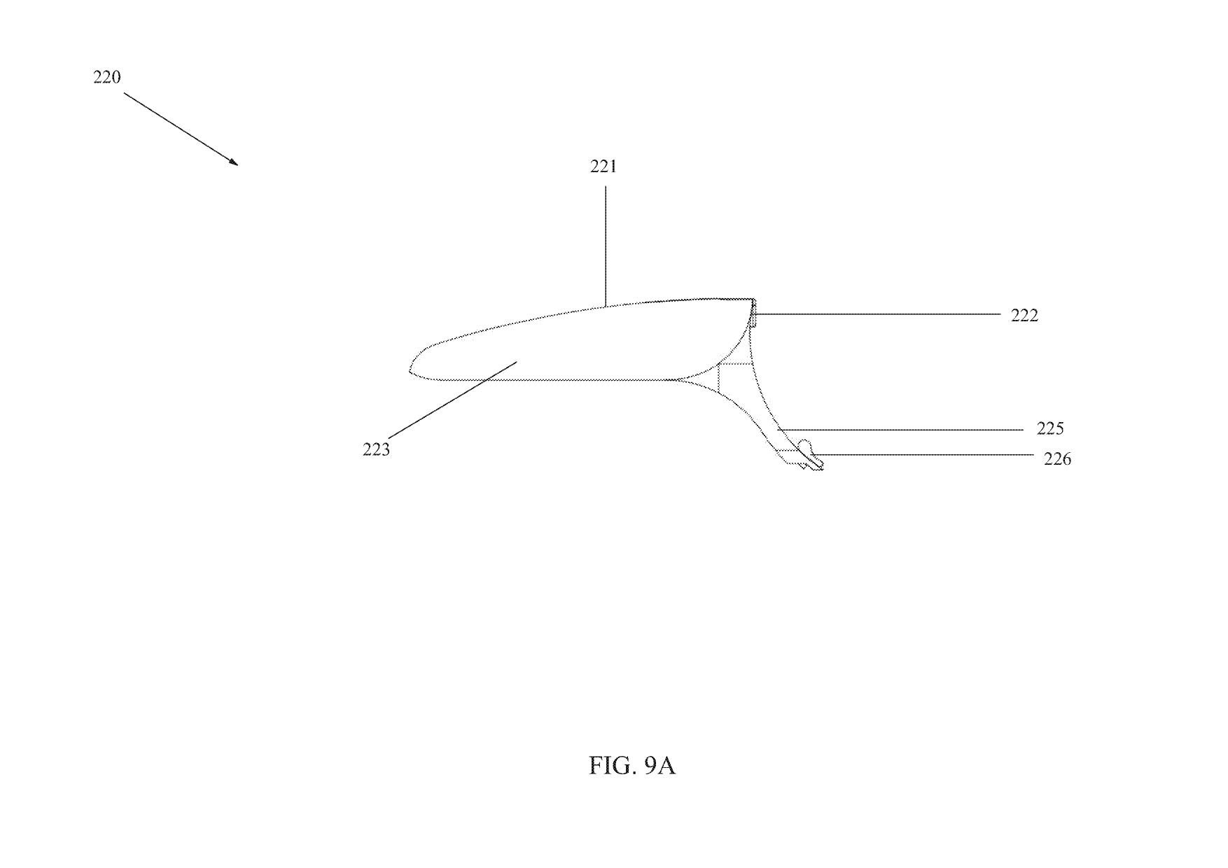

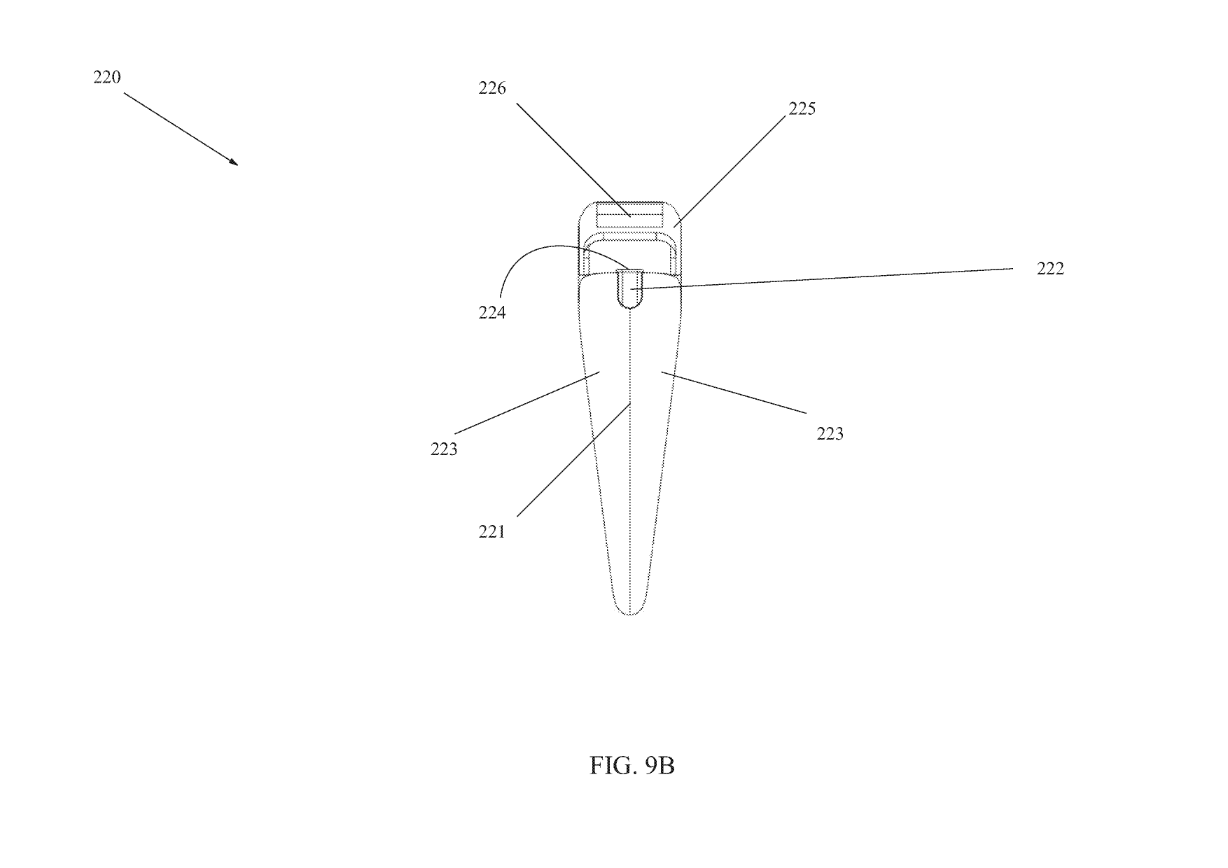

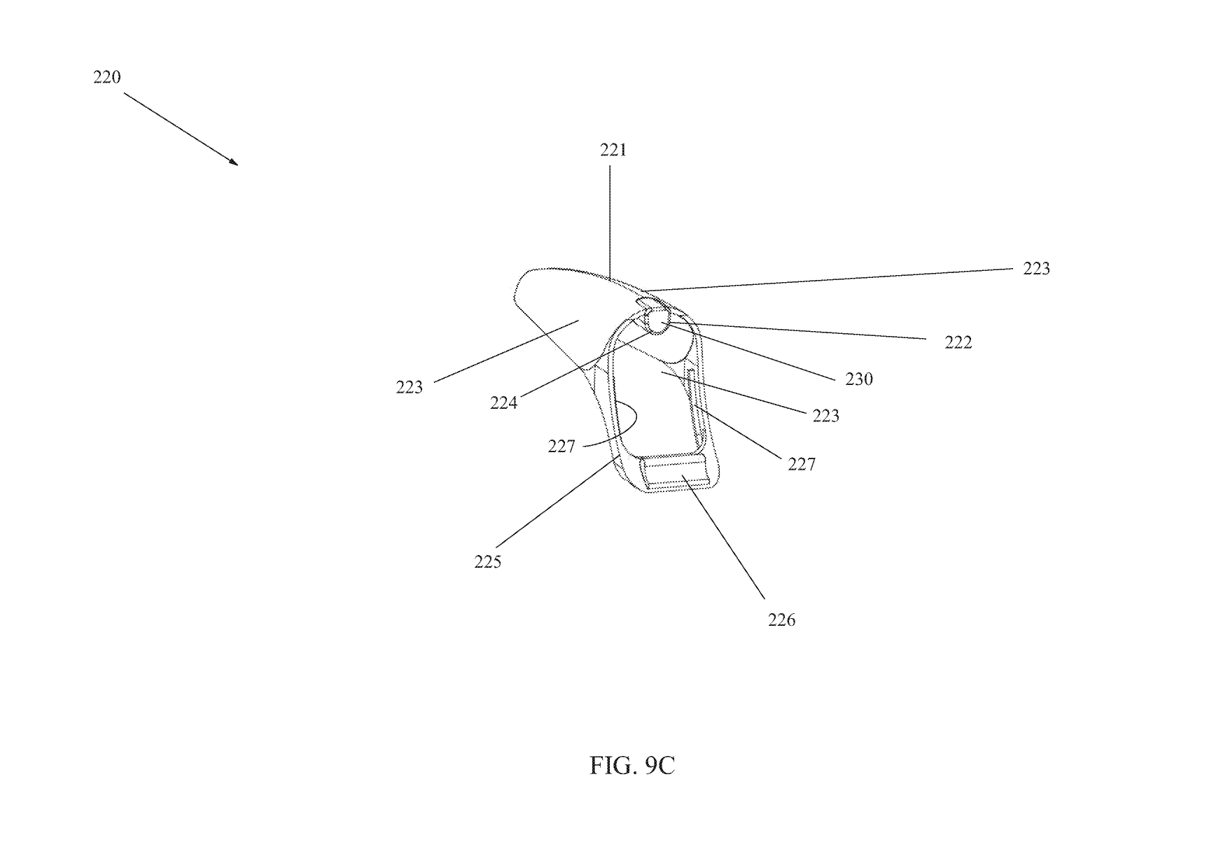

FIG. 9A is side perspective view of an anterior portion 220 of a speculum 200 in accordance with some embodiments.

FIG. 9B is an elevational top view of an anterior portion 220 of a speculum 200 in accordance with some embodiments.

FIG. 9C is a rear perspective view of an anterior portion 220 of a speculum 200 in accordance with some embodiments.

FIGS. 8A through 9C illustrate the posterior portion 210 and the anterior portion 220 as individual components, respectively. The posterior portion 210 and the anterior portion 220 have been described above, and will not be repeated for sake of brevity. Referring to FIG. 9C, the anterior portion 220 further includes a plurality of connecting rails 227, but is not limited thereto. A first of the plurality of connecting rails 227 is disposed on a first side of the opened handle 226 and a second of the plurality of connecting rails is disposed on a second side of the opened handle 226 opposite with respect to the first side of the opened handle 226. As described above, each of the plurality of connecting rails 227 removably connect and move (i.e., slide) within the first track 241 and the second track 242. As such, the plurality of connecting rails 227 facilitate movement of the anterior portion 210 in the first linear direction or the second linear direction in response to an application of force.

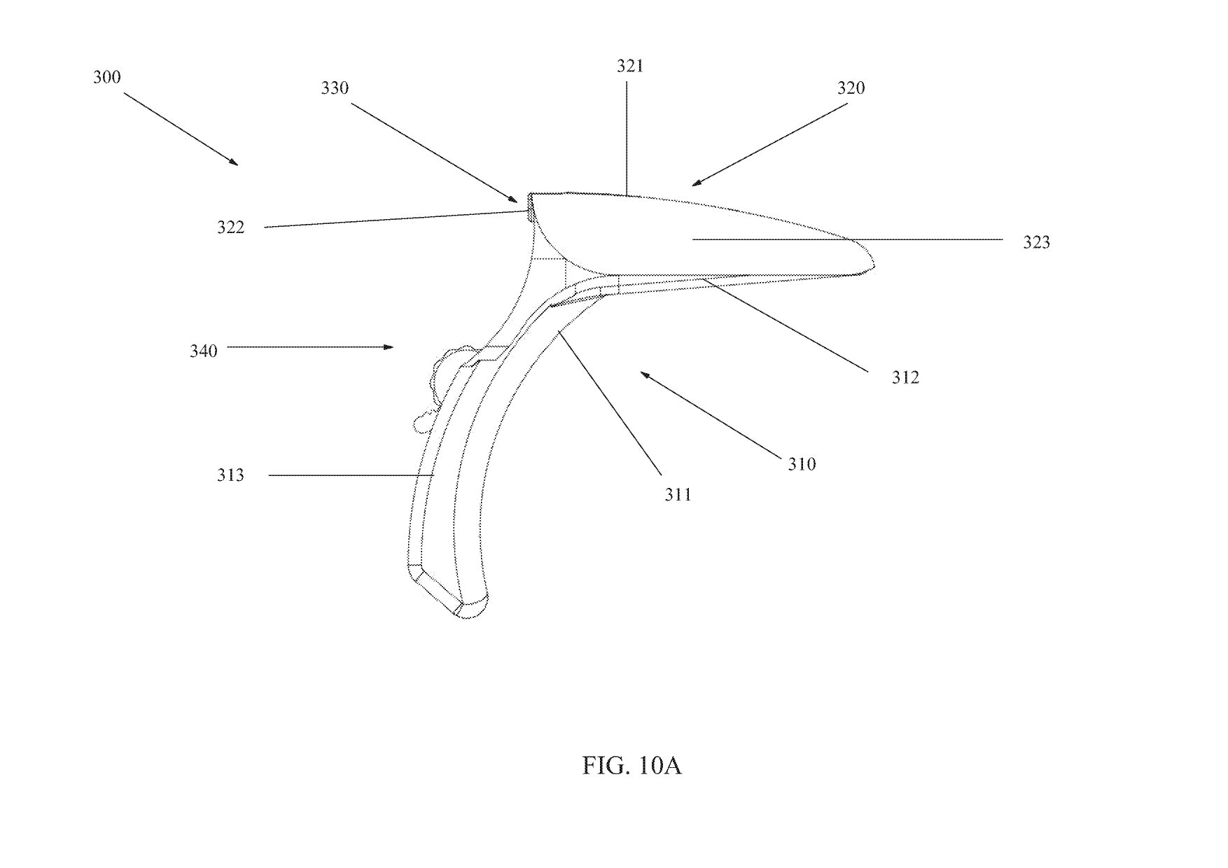

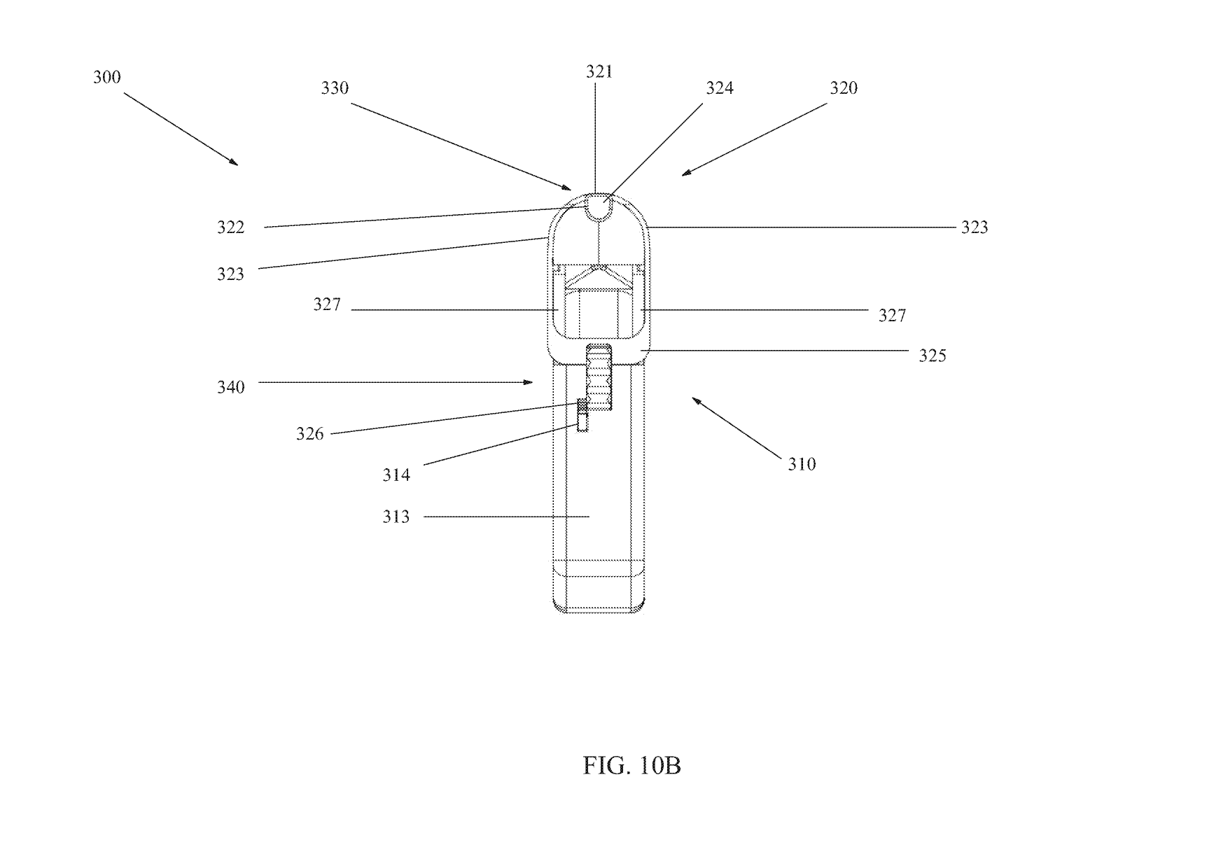

FIG. 10A is a side perspective view of a speculum 300 in accordance with some embodiments.

FIG. 10B is an elevational rear view of a speculum 300 in accordance with some embodiments. The speculum 300 may implement or be implemented by aspects of the speculum 100 or the speculum 200 as described with reference to FIGS. 3A through 9C. Similar to the speculum 100 and the speculum 200, the speculum 300 is a device that generally is used for medical examination of a patient. The speculum 300 is removably inserted into a vagina of a patient. More specifically, for example, the speculum 300 is inserted in a closed position into the vagina and moved to an opened position to move vaginal walls apart, expanding the vagina, and facilitate viewing of a cervix. In some embodiments, the speculum 300 includes disposable specula made of plastics, reusable specula made of metal, and/or reusable specula made of metal and overlaid with plastics or silicones, or a combination thereof.

Further, the speculum 300 is preferably made of materials that can withstand high-level disinfection that involve the use of chemicals such as a combination of bleach and hydrogen peroxide or a blend of peracetic acid and hydrogen peroxide. Accordingly, the speculum 300 is preferably made of at least one of the following materials: hard plastic (such as methacrylate, polycarbonate, polyvinyl chloride, polyethylene terephthalate glycol, acrylonitrile butadiene styrene) silicone, polymer, acrylic, ceramics, and plastic resins, etc.

Further, for some patients, the speculum 300 made of the above-mentioned materials may not be sturdy and strong enough to open the vaginal canal. Said speculum 300 may require a stronger metal or other material as a base and overlaid by the silicone or softer material. Thus, in different embodiments, the speculum 300 may preferably contain metal, such as stainless steel, and involve over with additional materials other processes including but not exclusive of two-shot molding, co-injection molding, insert molding, hybrid molding, and soft overmolding.

Referring to FIGS. 10A and 10B, in some embodiments, the speculum 300 includes a posterior portion 310, an anterior portion 320, a light source 330, and a connector 340, but is not limited thereto. The posterior portion 310 forms a handle and at least a portion thereof is inserted into the orifice. The anterior portion 320 is removably connected to the posterior portion 310 and at least a portion is inserted into the orifice with a portion of the posterior portion 310. The connector 340 connects the posterior portion 310 to the anterior portion 320. Moreover, the anterior portion 320 moves (i.e., slides, shifts) in a first linear direction away from the posterior portion 310 or a second linear direction toward the posterior portion 310 in response to an external application of force (e.g., pushing, pulling).

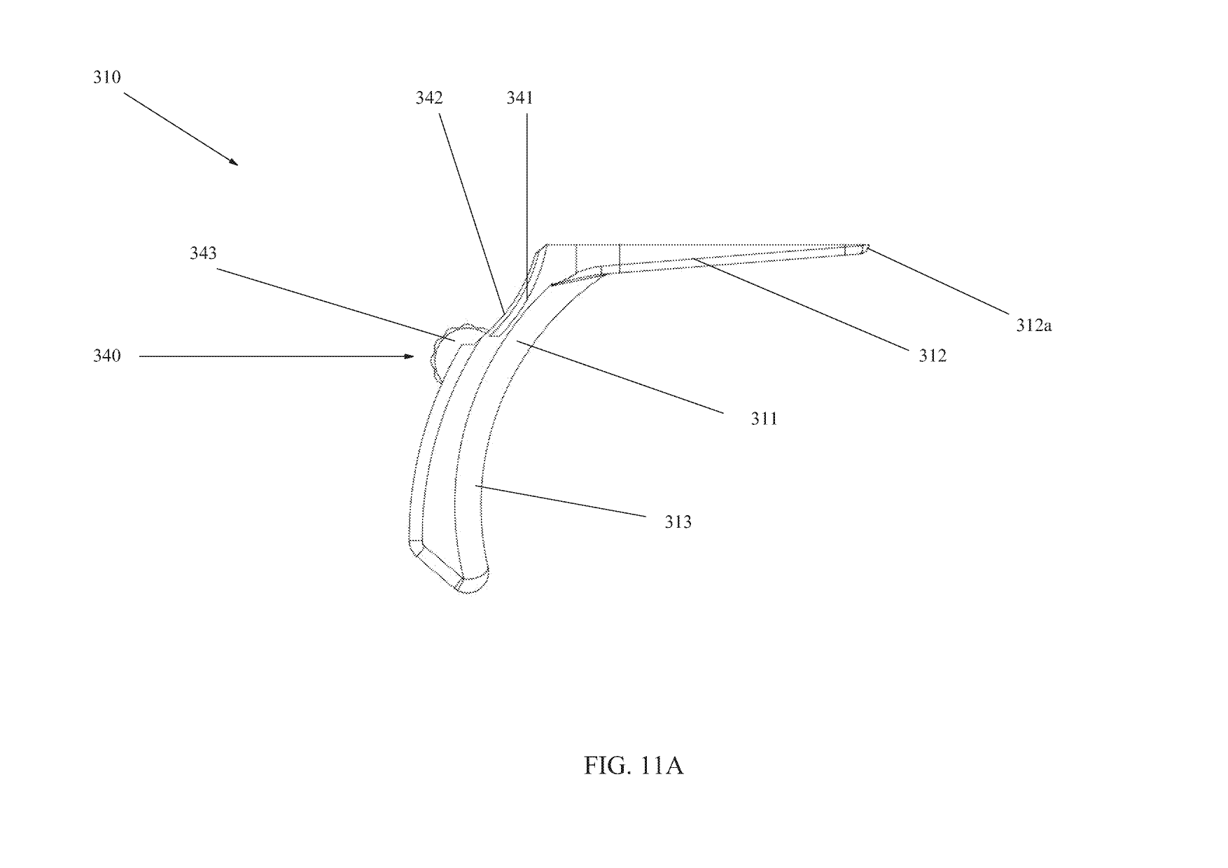

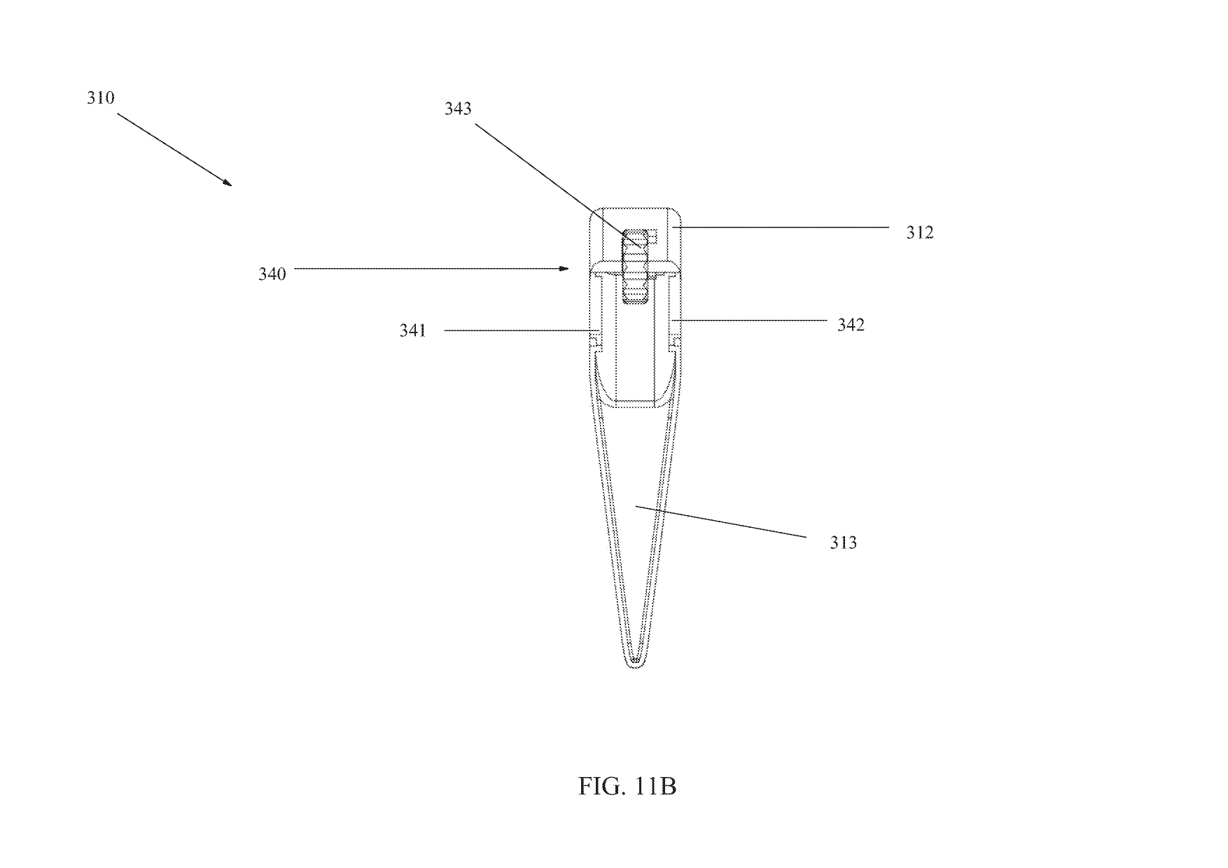

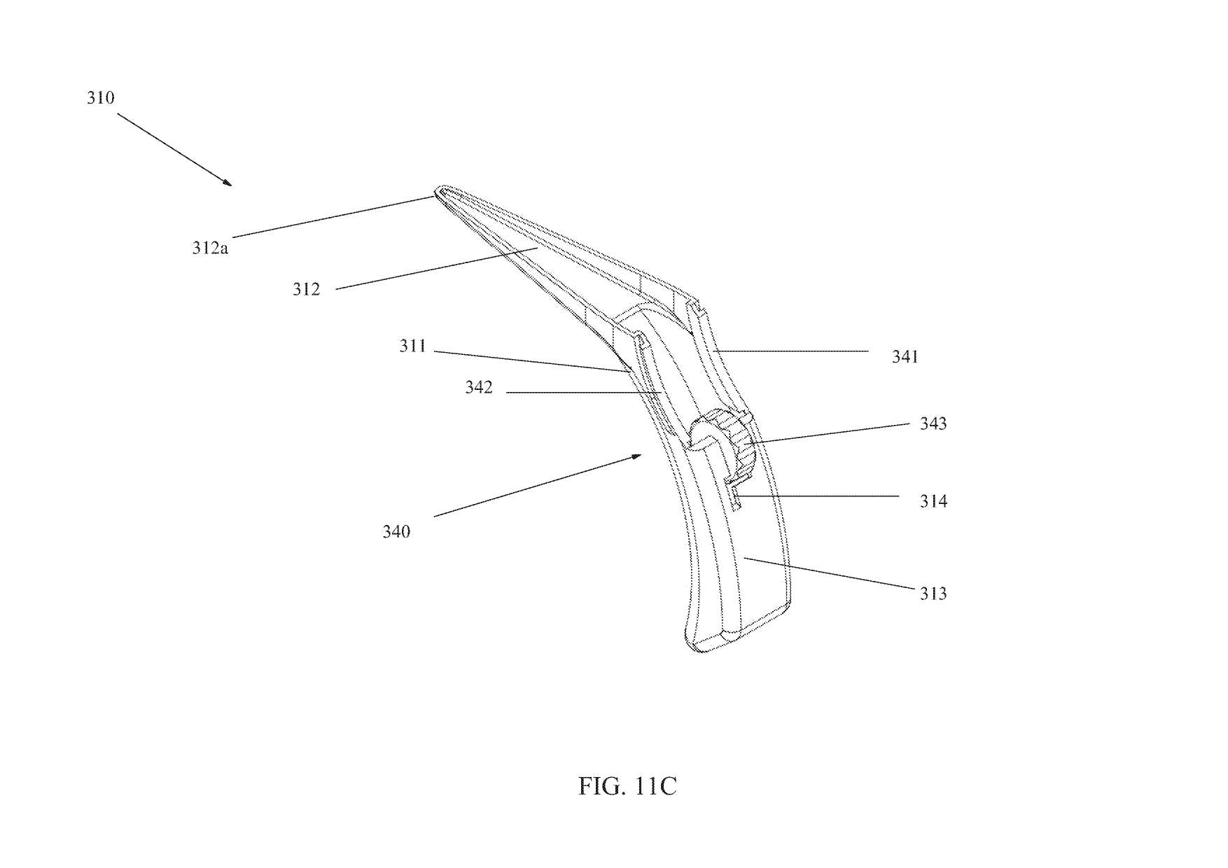

FIG. 11A is side perspective view of a posterior portion 310 of a speculum 300 in accordance with some embodiments.

FIG. 11B is an elevational top view of a posterior portion 310 of a speculum 300 in accordance with some embodiments.

FIG. 11C is a rear perspective view of a posterior portion 310 of a speculum 300 in accordance with some embodiments. In some embodiments, the posterior portion 310 includes a curved elongate body 311, a posterior blade 312, a handle 313, and a protrusion receiving opening 314, but is not limited thereto. The curved elongate body 311 is constructed in an arcuate shape (e.g., a C-shape). In contrast, prior speculum handles do not have any curved shape, let alone, a C-shape.

The posterior blade 312 is disposed at a first end of the curved elongate body 311. The posterior blade 312 extends away from the first end of the curved elongate body 311 with respect to a linear direction. In application, the posterior blade 312 facilitates insertion thereof into the orifice (e.g., vagina) of the patient. The handle 313 is disposed at a second end of the curved elongate body 311. The handle 313 is elongate and facilitates gripping thereof. In other words, the handle 313 is used to grip the posterior portion 310. In the depicted example of FIGS. 11A and 11C, the handle 313 is larger (i.e., greater size in length, width, depth) than the curved elongate body 311. However, in different embodiments, the handle 313 is similar in size to the curved elongate body 311, depending on construction. The posterior blade 312 connects to the handle 313 with a greater than ninety degree (90°) angle. For example, in some embodiments, the posterior blade 312 connects to the handle 313 between 110 to 130 degrees. The angle facilitates visualization during operation by preventing tissue impediment, such as from buttocks of the patient. In other words, the increased angle between the posterior blade 312 and/or the handle 313 prevent the buttocks from obstructing view of the vagina and the cervix. It also helps when the patient is on a gurney. The handle 313 is curved to prevent abutting against the buttocks and a rectum of the patient.

In the depicted example of FIG. 11C, the protrusion receiving opening 314 is disposed on at least a portion of the handle 313. In particular, the protrusion receiving opening 314 facilitates movement (i.e., rotation) of the connector 340. That is, a. portion of the connector 340 moves within the protrusion receiving opening 314.

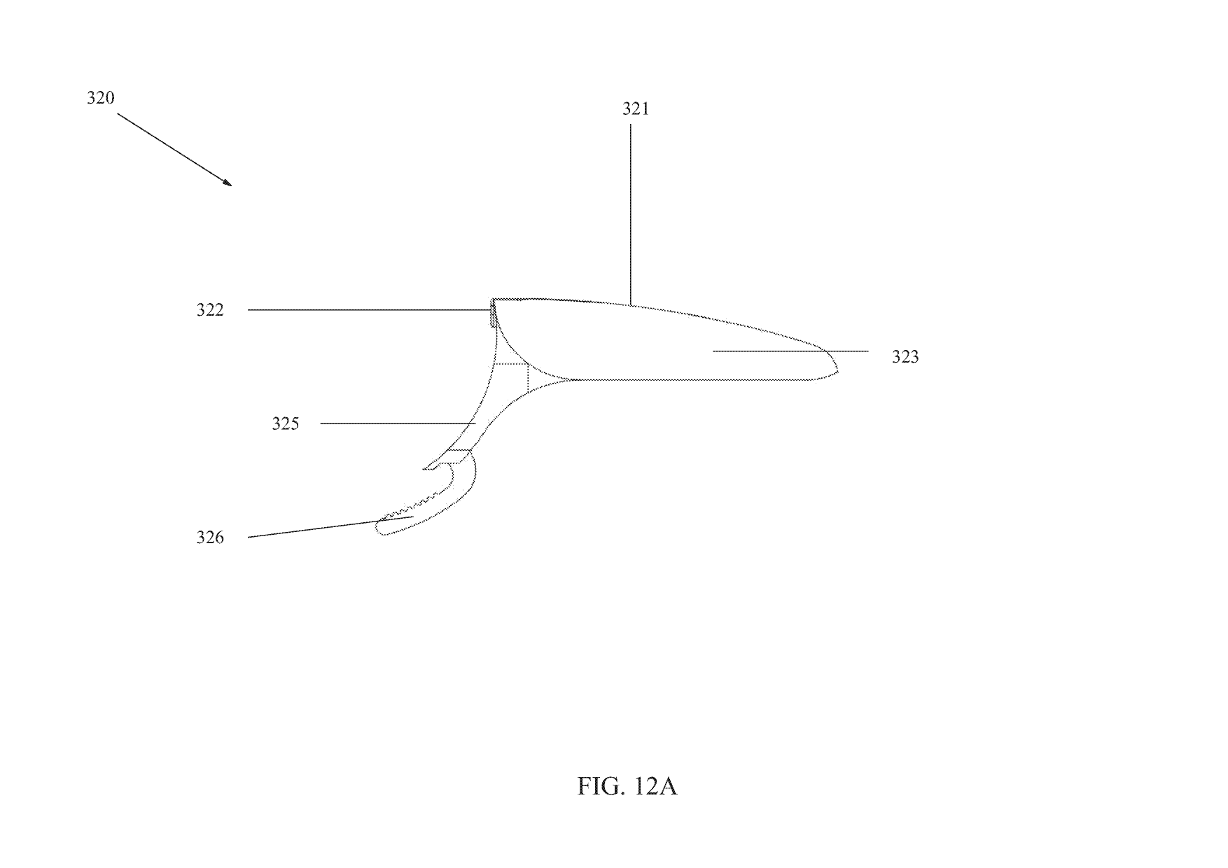

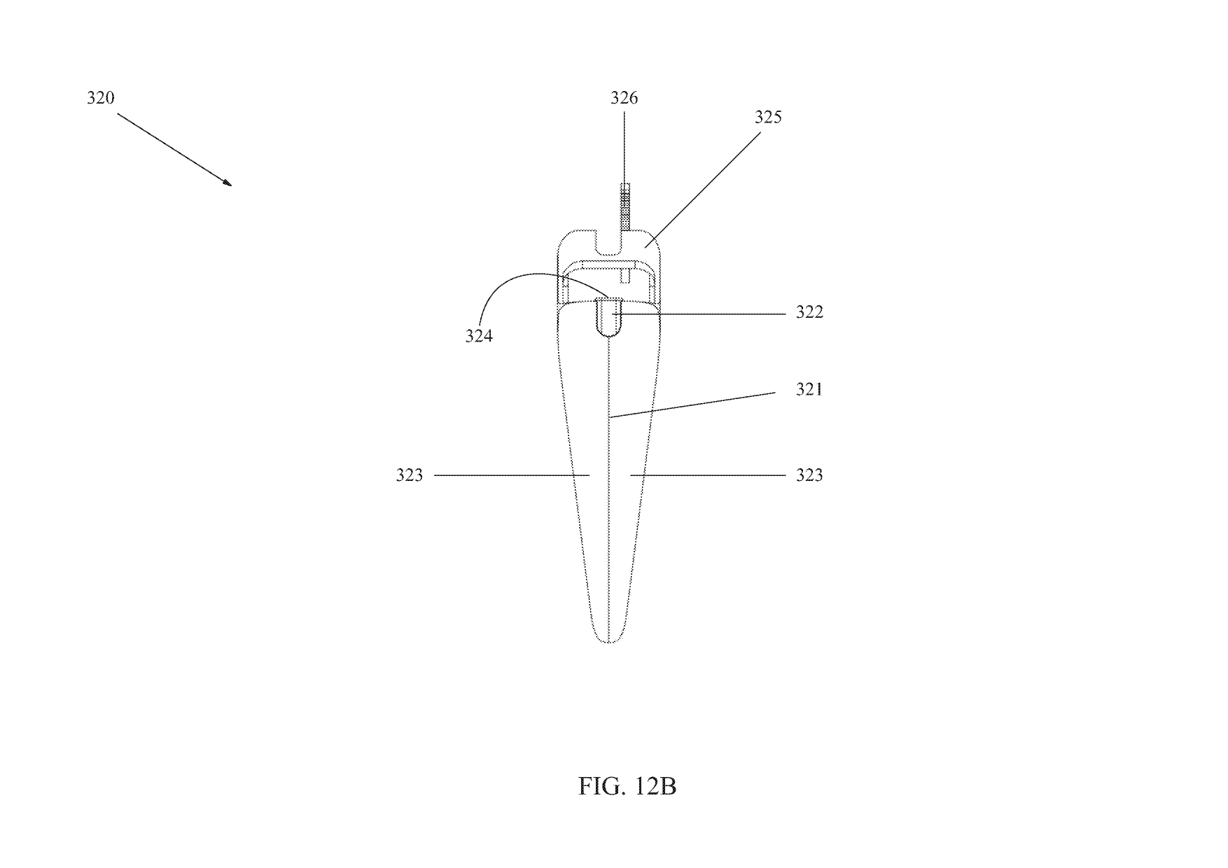

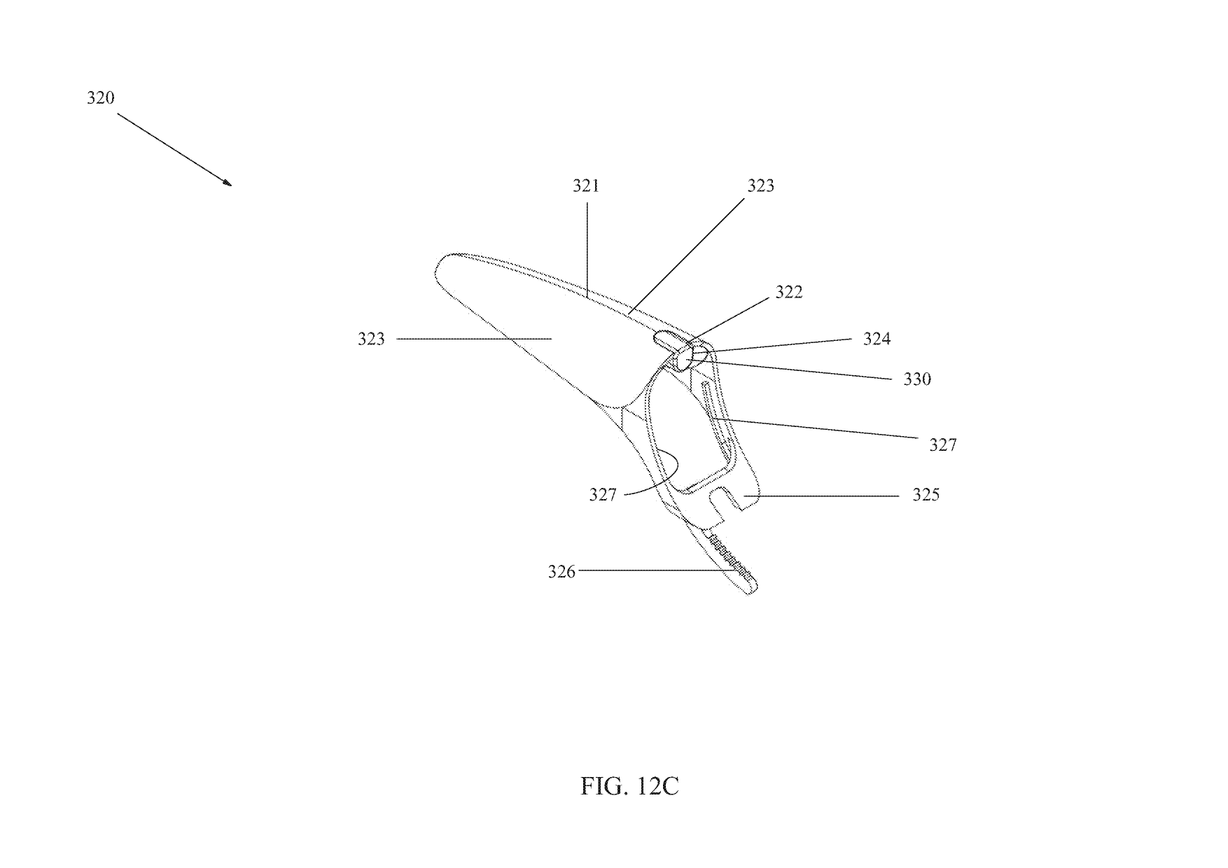

FIG. 12A is side perspective view of an anterior portion 320 of a speculum 300 in accordance with some embodiments.

FIG. 12B is an elevational top view of an anterior portion 320 of a speculum 300 in accordance with some embodiments.

FIG. 12C is a rear perspective view of an anterior portion 320 of a speculum 300 in accordance with some embodiments. In some embodiments, the anterior portion 320 includes an anterior blade 321, a light attachment portion 322, a plurality of side walls 323, and a channel 324, but is not limited thereto. The anterior blade 321 is constructed with an elongate shape. Referring to FIGS. 12A through 12C, it is important to note that the anterior blade 321 tapers from a first end (i.e., rear) toward a second end (i.e., front) opposite to the first end. Similarly, the posterior blade 312 tapers from a first end (e.g., rear) toward a second end (i.e., front) opposite to the first end. As such, the second end of the posterior blade 312 and the second end of the anterior blade 321 are narrower with respect to the first end of the posterior blade 312 and the first end of the anterior blade 321, respectively. In this manner, the anterior blade 321 improves comfort for the patient during insertion into the vagina, being narrower at the initial insertion point, while the larger (i.e., wider) first end, back of the speculum 300 improves visualization of the vagina and the cervix during operation by a medical professional by providing a wide viewing area and for passage of instruments as for a biopsy while at the same time, minimizing patient discomfort by narrowing the majority of the speculum 300 inside the vagina.

Referring to FIGS. 11A and 11C, in some embodiments, the posterior blade 312 includes a rounded tip 312a, but is not limited thereto. The rounded tip 312a curves in a direction. In particular, and preferably, the rounded tip 312a is disposed to curve toward the anterior blade 321. In some embodiments, the anterior blade 321 has a similar size (e.g., length, width, depth) as a size (e.g., length, width, depth) of the posterior blade 312. However, in different embodiments, the size of the anterior blade 321 is larger or smaller than the size of the posterior blade 312 based on different construction. In application, the anterior blade 321 facilitates insertion thereof into the orifice (e.g., vagina) of the patient. Collectively, the posterior blade 312 and the anterior blade 321 facilitate insertion into the vagina of the patient. Furthermore, the anterior blade 321 moves at least a portion of the vagina in response to moving in the first linear direction or the second linear direction, as described above. In particular, the anterior blade 321 expands the vagina in response to moving in the first linear direction or the second linear direction, such that movement of the anterior blade 321 within the vagina facilitates access therein and access to the cervix of the patient, such as during medical operations (e.g., surgery). The posterior blade 312 and/or the anterior blade 321 are preferably two smooth and curved metal or plastic blades. The user clinician inserts the posterior blade 312 and/or the anterior blade 321 into the vagina while closed and then gently moves the anterior blade 321 away from (i.e., separates) the posterior blade 312 to hold the vaginal walls apart, providing a clear view of the cervix. The posterior blade 312 and/or the anterior blade 321 preferably have rounded and polished edges to minimize discomfort and risk of tissue injury during insertion and removal of the speculum 300. Therefore, the posterior blade 312 and/or the anterior blade 321 come together at the front, tapered portion with no exposed edges to provide aerodynamic entry into the vagina and minimize the need to spread the labia during insertion and minimize the risk of pinching tissue.

In the depicted example of FIGS. 12A through 12C, the light attachment portion 322 is disposed on at least a portion of the anterior blade 321 and/or within a portion of the anterior blade 321. The plurality of side walls 323 are disposed on at least a portion of the anterior blade 321. More specifically, a first side wall of the plurality of side walls 323 is disposed on at least a portion of a first side of the anterior blade 321 and extends away from the first side of the anterior blade 321 with respect to a first direction (i.e., a first angle from a vertex of the anterior blade 321), and a second side wall of the plurality of side walls 323 is disposed on at least a portion of a second side of the anterior blade 321 and extends away from the second side of the anterior blade 321 with respect to a second direction (i.e., a second angle from a vertex of the anterior blade 321) different from the first direction. However, in some embodiments, the first side wall of the plurality of side walls 323 and the second side wall of the plurality of side walls 323 extend away from the first side of the anterior blade 321 and the second side of the anterior blade 321 in a same direction. Referring again to FIGS. 12A through 12C, each of the plurality of side walls 323 extend away from the anterior blade 321 toward the posterior blade 312. As such, the plurality of side walls 323 at least partially obstruct an area between the plurality of side walls 323 and the posterior blade 312. Accordingly, the plurality of side walls 323 prevent an external object, such as body tissue within the vagina of the patient from entering the area between the plurality of side walls 323 and/or the posterior blade 312. In conventional specula, the anterior blade 321 and/or the posterior blade 312 are flat or have slight curvature similar to a spoon. In contrast, the anterior blade 321 is curved like “C” shape, such that the plurality of side walls 323 are sloped to facilitate retraction during insertion into the vagina. During medical use, the plurality of side walls 323 are sloped to facilitate retraction of the vagina without moving the posterior blade 312 and/or the anterior blade 321. In other words, the plurality of side walls 323 separate tissue within the vagina during insertion of the speculum 300 without moving (i.e., separating) the anterior blade 321 away from the posterior blade 312.

As described above, the anterior portion 320 includes the plurality of side walls 323 and extend away from the anterior blade 321. Alternatively, and/or in addition thereto, in different embodiments, the posterior portion 310 includes the plurality of side walls 323. For example, the plurality of side walls 323 are disposed on a first side of the posterior blade 312 and a second side of the posterior blade, and each of the plurality of side walls 323 on the posterior blade 312 extend away from the posterior blade 312 toward the anterior blade 321. Similarly, the plurality of side walls 323 at least partially obstruct the area between the plurality of side walls 323 and the anterior blade 321. Accordingly, the plurality of side walls 323 prevent the external object, such as body tissue within the vagina of the patient from entering the area between the plurality of side walls 323 and/or the anterior blade 321.

Referring to FIGS. 12B and 12C, the channel 324 is disposed within at least a portion of the light attachment portion 322. The channel 324 extends from a first end of the light attachment portion 322 toward a second end of the light attachment portion 322. Also, the channel 324 extends from a first end of the anterior blade 321 toward a second end of the anterior blade 321. In other words, the channel 324 is elongate and, in some embodiments, is cylindrical in shape. However, in different embodiments, the shape of the channel 324 is rectangular, triangular, pentagonal, hexagonal, heptagonal, or octagonal.

In some embodiments, the light source 330 is a micro light-emitting diode (LED). However, in different embodiments, the light source 330 is an incandescent light, a fluorescent bulb, a xenon blub, and the like. In some embodiments, the light source 330 includes an elongated rod, a square, and a rectangle, but is not limited thereto. In different embodiments, the light source 330 includes mini LED, organic LED, and other light sources that are small, energy-efficient, and can be manually switched on and off. Further, in different embodiments, the light source 330 can be configured to be removably attached to the anterior blade 321. In other words, the posterior blade 312 and/or the anterior blade 321 in different embodiments may be free of the light attachment portion 322 and can be detachably coupled with the light source 330. In different embodiments, the anterior blade 321 is separate, independent, detachably coupled light source 330 that is inserted.

The light source 330 detachably connects (i.e., inserts) within the channel 324 of the light attachment portion 322 and extends from the first end of the channel 324 toward the second end of the channel 324. It is important to note that the light source 330 is detachable from the channel 324 in absence of an application of external force from a tool (e.g., screwdriver, wrench, hammer, etc.). In other words, the light source 330 is detachable using only hands or other limb of a user. The light source 330 is enclosed within a boundary of the channel 324 after connecting the light source 330 within the channel 324. The light source 330 at least partially illuminates an area between the posterior blade 312 (i.e., posterior portion 310) and the anterior blade 321 (i.e., anterior portion 320) in response to being turned on. The light source 330 provides illumination for example, the vaginal cavity and specifically illuminates the vaginal walls and cervix for better visualization during examination. Because the light source is on the anterior blade, it is not obscured by blood, tissue or other body fluid running out of the vagina along the posterior blade. Additionally, because the light is inserted along the anterior blade, it provides more direct illumination of the cervix and distal vagina. Conversely, the light source 330 is prevented from illuminating in response to being turned off.

Referring to FIGS. 11A through 11C, the connector 340 includes a first track 341, a second track 342, and a control wheel 343, but is not limited thereto. The first track 341 is disposed on at least a portion of a first side of the curved elongate body 311. The second track 342 is disposed on at least a portion of a second side of the curved elongate body 311 opposite to the first side of the curved elongate body 311. Moreover, the control wheel 343 is movably (i.e., rotatably) disposed on at least a portion of the curved elongate body 311. The first track 341 and the second track 342 receive a corresponding connecting rail therein. The connecting rails are disposed on at least a portion of the anterior portion 320.

In the depicted example of FIGS. 12A through 12C, the anterior portion 320 further includes an opened handle 325 and a ridged blade 326, but is not limited thereto. The opened handle 325 is angularly disposed at a rear end of the anterior blade 321. The opened handle 325 extends away from the rear end of the anterior blade 321 with respect to a direction. The opened handle 225 includes an opening to facilitate viewing an area between the posterior blade 312 and/or the anterior blade 311. As such, the opened handle 325 facilitates viewing of an interior of the vagina and/or the cervix while inserted into the vagina. The ridged blade 326 is disposed on at least a portion of the opened handle 225 and linearly extends away from the opened handle with respect to the direction. In the depicted example of FIG. 10A, the ridged blade 326 connects to the control wheel 343, such that the control wheel 343 moves the anterior portion 320. To illustrate via an example, the anterior portion 320 moves (i.e., slides) in the first linear direction away from the posterior portion 310 in response to the control wheel 343 moving (i.e., rotating) in a first rotational direction (i.e., clockwise) or a second rotational direction (i.e., counterclockwise). Conversely, the anterior portion 320 moves in the second linear direction toward the posterior portion 310 in response to the control wheel 343 moving in the second rotational direction or the first rotational direction. Thus, the control wheel 343 adjusts positioning of the anterior blade 321 with respect to the posterior blade 312 to move tissue (e.g., vagina) and facilitate visualization in the vagina and/or the cervix.

Referring to FIG. 12C, the anterior portion 320 further includes a plurality of connecting rails 327, but is not limited thereto. A first of the plurality of connecting rails 327 is disposed on a first side of the opened handle 326 and a second of the plurality of connecting rails is disposed on a second side of the opened handle 326 opposite with respect to the first side of the opened handle 326. As described above, each of the plurality of connecting rails 327 removably connect and move (i.e., slide) within the first track 341 and the second track 342. As such, the plurality of connecting rails 327 facilitate movement of the anterior portion 310 in the first linear direction or the second linear direction in response to movement of the control wheel 343.

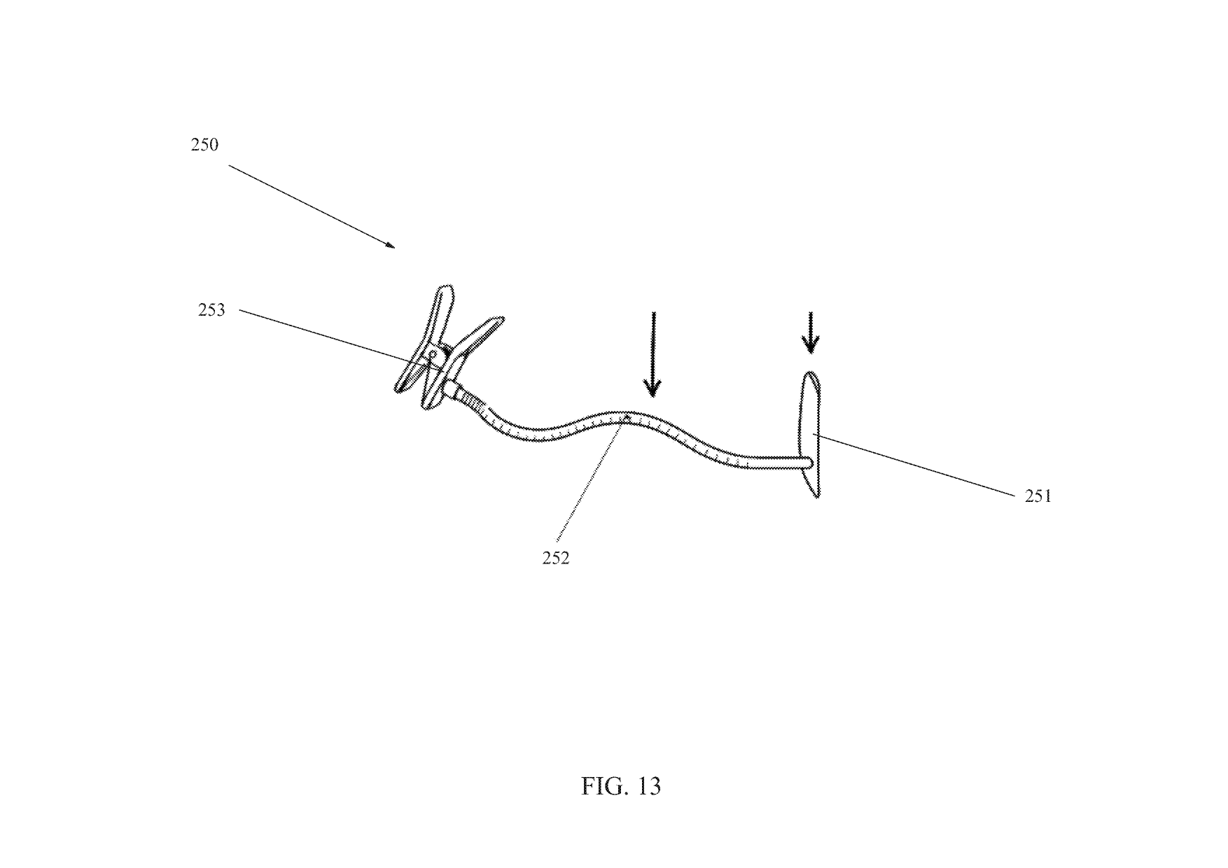

FIG. 13 is a side perspective of a viewing device 250 disposed on a speculum 200 in accordance with some embodiments. In some embodiments, the viewing device 250 includes a mirror 251, a flexible rod 252, and a connecting fastener 253, but is not limited thereto. The mirror 251 reflects objects and light rays thereon to facilitate visualization. In some embodiments, the flexible rod 252 is an elongate rod and at least partially deforms (i.e., bends, twists, expands, collapses) in response to an application of force (e.g., pushing, pulling) thereon. In other words, the flexible rod 252 is moved and shaped based on bending or twisting to position the flexible rod 252. After movement, the flexible rod 252 retains a position. For example, bending the flexible rod 252 into an L-shape or an S-shape will retain the L-shape or the S-shape until the flexible rod 252 is adjusted into a different shape or position. As such, for example, the mirror 251 is used during operation to allow the patient to visualize what the medical professional may see regarding the vagina and/or the cervix. In the depicted example of FIG. 8, the connecting fastener 253 includes a clip. However, in different embodiments, the connecting fastener 253 includes an adhesive (e.g., tape), a band, a magnet, and/or any combination thereof, but is not limited thereto. The connecting fastener 253 removably connects the flexible rod 252 to the speculum 200 or the speculum 100.

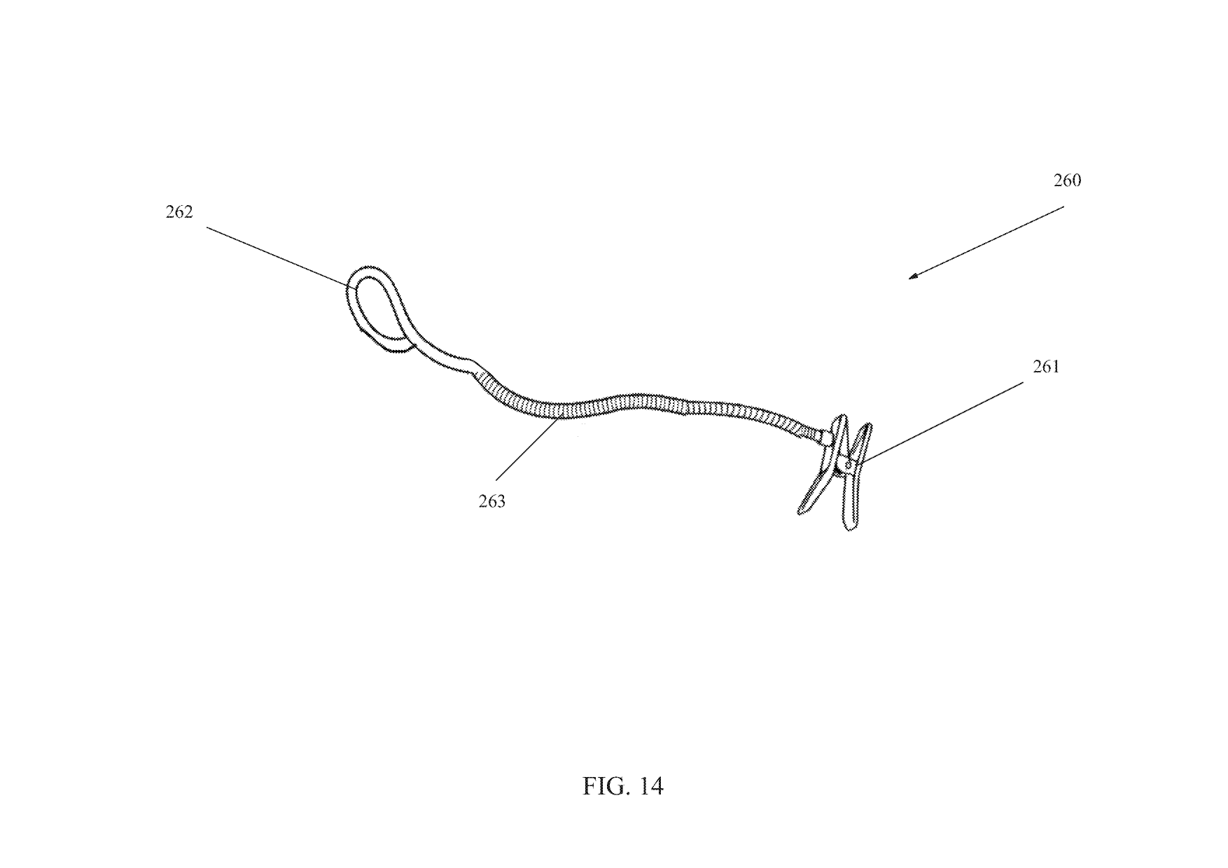

FIG. 14 is a side perspective of a holding device 260 disposed on a speculum 200 in accordance with some embodiments. In some embodiments, the holding device 260 includes a speculum fastener 261, a handle 262, and an extension rod 263, but is not limited thereto. In some embodiments, the speculum fastener 261 includes a clip, an adhesive (e.g., tape), a band, a magnet, and/or any combination thereof, but is not limited thereto. The speculum fastener 261 removably connects to at least a portion of the speculum 200 or the speculum 100. The handle 262 facilitates gripping thereof. The extension rod 263 is disposed on the speculum fastener 261 at a first end and on the handle 262 at a second end. In other words, the speculum fastener 261 is disposed at the first end of the extension rod 263 and the handle 262 is disposed at the second end of the extension rod 263. Therefore, the speculum 200 moves in response to movement of the handle 262 while the speculum 200 is connected to the speculum fastener 261. In this manner, a user, such as the patient moves the speculum 200 as desired. The holding device 260 is used, for example, by the patient with emotional and/or mental trauma to facilitate insertion of the speculum 200 into the vagina during medical procedure and/or gynecological exam.

The foregoing descriptions of specific implementations have been presented for purposes of illustration and description. They are not intended to be exhaustive or to limit the invention to the precise forms disclosed, and modifications and variations are possible in view of the above teaching. The exemplary implementations were chosen and described to best explain the principles of the invention and its practical application, to thereby enable others skilled in the art to best utilize the invention and its implementations with modifications as suited to the use contemplated.

It is therefore submitted that the invention has been shown and described in the most practical and exemplary implementations. It should be recognized that departures may be made which fall within the scope of the invention. With respect to the description provided herein, it is submitted that the optimal features of the invention include variations in size, materials, shape, form, function, manner of operation, assembly, and use. All structures, functions, and relationships equivalent or essentially equivalent to those disclosed are intended to be encompassed by the invention.

1. A speculum, comprising:
a posterior portion to facilitate gripping thereof;
an anterior portion movably connected to the posterior portion, the anterior portion moving in at least one of a first linear direction and a second linear direction; and
a light source detachably connected within at least a portion of the anterior portion and at least partially illuminating an area between the posterior portion and the anterior portion in response to being turned on.

2. The speculum of claim 1, wherein the posterior portion comprises:
a curved elongate body;
a posterior blade disposed at a first end of the curved elongate body to extend away from the curved elongate body; and
a handle disposed at a second end of the curved elongate body to facilitate gripping thereof.

3. The speculum of claim 2, wherein an angle of the curved elongate body between the posterior blade and the handle exceeds ninety degrees.

4. The speculum of claim 1, wherein a rear of the posterior portion and a rear of the anterior portion taper toward a front of the posterior portion and a front of the anterior portion.

5. The speculum of claim 1, wherein the anterior portion comprises:
an anterior blade having an elongate shape;
a light attachment portion disposed within at least a portion of the anterior blade to receive the light source therein; and
a plurality of side walls, each of the plurality of side walls disposed on at least a portion of a first side of the anterior blade and a second side of the anterior blade, and a first of the plurality of side walls curvedly extending away from the first side of the anterior blade with respect to a first direction and a second of the plurality of side walls curvedly extending away from the second side of the anterior blade with respect to a second direction.

6. The speculum of claim 5, wherein the anterior portion further comprises:
an opened handle disposed on at least a portion of the anterior blade and extending away from the anterior blade with respect to a direction; and
a slide lock movably disposed on at least a portion of the opened handle to prevent movement of the anterior portion in a first position and facilitate movement of the anterior portion in at least one of the first linear direction away from the posterior portion and the second linear direction toward the posterior portion.

7. The speculum of claim 1, further comprising a connector connected at a first end to the anterior portion and connected at a second end to the posterior portion.

8. The speculum of claim 7, wherein the anterior portion moves at least partially away from the posterior portion in the first linear direction via the connector, and moves at least partially toward the posterior portion in the second linear direction via the connector.

9. The speculum of claim 7, wherein the connector comprises a control wheel movably disposed on at least a portion of the anterior portion.

10. The speculum of claim 9, wherein the anterior portion comprises:
an opened handle disposed on at least a portion of the anterior blade and extending away from the anterior blade with respect to a direction; and
a ridged blade disposed on at least a portion of the opened handle and linearly extending away from the opened handle with respect to the direction, the ridged blade to connect to the control wheel to move the anterior portion in at least one of the first linear direction or the second linear direction in response to movement of the control wheel against the ridged blade.

11. A speculum, comprising:
a posterior portion constructed of materials that can withstand a high-level disinfection and facilitates gripping thereof;
an anterior portion constructed of materials that can withstand the high-level disinfection and movably connected to the posterior portion, the anterior portion moving in a first rotational direction or a second rotational direction; and
a light source detachably connected within at least a portion of the anterior portion and at least partially illuminating an area between the posterior portion and the anterior portion in response to being turned on.

12. The speculum of claim 11, wherein the posterior portion and the anterior portion prevent damage from at least one of bleach, hydrogen peroxide, and a combination of peracetic acid and hydrogen peroxide based on the materials.

13. The speculum of claim 11, wherein the posterior portion and the anterior portion use the materials that comprise at least one of hard plastic, silicone, polymer, acrylic, ceramics, plastic resins, and metals.

14. The speculum of claim 11, wherein the posterior portion and the anterior portion are constructed of a first material that overlays a second material, the first material is at least one of a plastic, a silicone, a hard plastic, a polymer, an acrylic, a ceramic, and plastic resin, and the second material is a metal.

15. A speculum, comprising:
a posterior portion to facilitate gripping thereof;
an anterior portion movably connected to the posterior portion, the anterior portion moving in at least one of a first linear direction and a second linear direction; and
a light source detachably connected within at least a portion of the anterior portion and extending from a first end of anterior portion toward a second end of the anterior portion, the light source at least partially illuminating an area between the posterior portion and the anterior portion in response to being turned on.

16. The speculum of claim 15, wherein the light source is at least one of an elongated rod, a square, and a rectangle.

17. The speculum of claim 15, wherein the light source detachably connects within a channel extending from the first end of the anterior portion toward the second end of the anterior portion.

18. The speculum of claim 17, wherein the light source is enclosed within a boundary of the channel after connecting the light source within the channel.

19. The speculum of claim 15, wherein the light source is a micro light-emitting diode (LED).

20. The speculum of claim 15, wherein the light source is detachable from the anterior portion in absence of application of external force from a tool.
